(19) United States
(12) Patent Application Publication
(10) Pub. No.: US 2025/0232571 A1
(43) Pub. Date: Jul. 17, 2025

(54) SYSTEMS, DEVICES, AND METHODS FOR LEVEL IDENTIFICATION OF THREE-DIMENSIONAL ANATOMICAL IMAGES

(71) Applicant: Augmedics, Inc., Arlington Heights, IL (US)

(72) Inventors: Krzysztof B. SIEMIONOW, Miami, FL (US); Cristian J. LUCIANO, San Diego, CA (US); Michal TRZMIEL, Warsaw (PL); Dominik GAWEL, Warsaw (PL); Edwing Isaac MEJIA OROZCO, Warsaw (PL)

(21) Appl. No.: 18/700,095

(22) PCT Filed: Oct. 17, 2022
(86) PCT No.: PCT/US2022/078225

§ 371 Date: Apr. 10, 2024

Related U.S. Application Data
(60) Provisional application No. 63/256,306, filed on Oct. 15, 2021.

(51) Int. Cl. G06V 10/82; A61B 34/10; G06V 10/25; G06V 10/26; G06V 10/764; G06V 20/64; G06V 20/70

(52) U.S. Cl. CPC G06V 10/82; G06V 10/25; G06V 10/26; G06V 10/764; G06V 20/64; G06V 20/70; A61B 34/10; A61B 2034/105; G06V 2201/033

(57) ABSTRACT

Embodiments include exemplary systems. methods. and computer-accessible medium for analysis of anatomical images and identification of anatomical components and/or structures. In some embodiments. systems. devices. and methods described herein relate to identification of levels of a spine and other anatomical components associated with those levels.

### CROSS-REFERENCE TO RELATED APPLICATIONS

This application claims priority to U.S. Provisional Application No. 63/256,306 entitled “LEVEL IDENTIFICATION OF THREE-DIMENSIONAL ANATOMICAL IMAGES,” filed Oct. 15, 2021, the entire disclosure of which is incorporated herein by reference.

### TECHNICAL FIELD

The present disclosure relates generally to systems, devices, and methods for identifying anatomical image data, and specifically relates to identifying levels of vertebrae in three-dimensional (3D) anatomical image data.

### BACKGROUND

A patient's spinal column is a complex system of bones and soft tissue structures. The spine, which forms part of the spinal column, functions as the body's central support structure, and is composed of many individual bones known as vertebrae. Intervertebral discs are positioned between adjacent vertebrae to provide support and cushioning between the vertebrae. The vertebrae and intervertebral discs, together with other soft tissue structures (e.g., ligaments, nervous systems structures, etc.) in their vicinity, form the spinal column. Each patient's spine varies in size and shape, with changes that can occur due to environmental factors, health, age, etc. A healthy spine can have certain predefined curves, but deformities can occur that can cause pain, e.g., via pinching of nerves and other soft tissue structures, as well as changes in those predefined curves.

There are various treatment options aimed at correcting spinal deformities. When operating in spinal anatomy, it can be important to identify the specific location, geometric limits, and other parameters associated with a surgical site. Software-based tools can be used, together with image data of patient anatomy, to assist physicians in pre-operative planning of surgical procedures in spinal anatomy. Image-guided or computer-assisted surgery can also be used to assist physicians in navigating to and/or operating on a target area of interest during a surgical procedure. Such systems and devices can analyze image data of patient anatomy acquired through one or more imaging systems, including, for example, computed tomography (CT), magnetic resonance imaging (MRI), X-ray, ultrasound, and fluoroscopy systems.

Traditional X-ray and CT are common methods for acquiring information of patient anatomy, including, for example, a spine of the patient. Traditional X-rays involve directing high-energy electromagnetic radiation at a patient's body, and capturing a resulting two-dimensional (2D) X-ray profile on a film or plate. X-ray imaging, however, can subject patients to high levels of radiation. Analysis of X-rays can also be subjective based on physician training and experience. Currently, these is no autonomous way to objectively analyze X-rays. Accordingly, performing necessary measurement on X-rays requires time and can be subject to user error. Lack of autonomous methods of analyzing X-rays also makes it difficult to objectively compare a patient's X-rays over time, e.g., to track a patient's progress. Due to these limitations, it is not presently possible to reliably predict certain outcomes based on X-ray imaging. It is also not presently possible to obtain necessary measurements in an autonomous and/or consistent fashion that ensures reliability and reproducibility of such measurements.

CT involves using controlled amounts of X-ray radiation to obtain 3D image data of patient anatomy. Existing CT systems can include a rotating gantry that has an X-ray tube mounted on one side and an arc-shaped detector mounted on an opposite side. An X-ray beam can be emitted in a fan shape as the rotating frame spins the X-ray tube and detector around a patient. Each time the X-ray tube and detector make a 360° rotation and the X-ray passes through the patient's body, an image of a thin section of the patient anatomy can be acquired. During each rotation, the detector can record about 1,000 images or profiles of the expanded X-ray beam. Each profile can then be reconstructed by a dedicated computer into a 3D image of the section that was scanned. Accordingly, CT systems use a collection of multiple 2D CT scans or X-rays to construct a 3D image of the patient anatomy. The speed of gantry rotation, along with slice thickness, contributes to the accuracy and/or usefulness of the final image. Commonly used intraoperative CT imaging systems have a variety of settings that allow for control of the radiation dose. In certain scenarios, high dose settings may be chosen to ensure adequate visualization of the anatomical structures. The downside is increased radiation exposure to the patient. The effective doses from diagnostic CT procedures are typically estimated to be in the range of 1 to 10 millisieverts (mSv). Such high doses can lead to increased risk of cancer and other health conditions. Low dose settings are therefore selected for CT scans whenever possible to minimize radiation exposure and associated risk of cancer development. Low dose settings, however, may have an impact on the quality of the image data available for the surgeon.

MRI imaging systems operate by forming a strong magnetic field around an area to be imaged. In most medical applications, protons (e.g., hydrogen atoms) in tissues containing water molecules produce a signal that is processed to form an image of the body. First, energy from an oscillating magnetic field is temporarily applied to the patient at an appropriate resonance frequency. The excited hydrogen atoms emit a radio frequency (RF) signal, which is measured by a RF system. The RF signal may be made to encode position information by varying the main magnetic field using gradient coils. As these coils are rapidly switched on and off, they product the characteristic repetitive noise of an MRI scan. Contrast between different tissues can be determined by the rate at which excited atoms return to their equilibrium state. In some instances, exogenous contrast agents may be given intravenously, orally, or intra-articularly, to further facilitate differentiation between different tissues. The major components of an MRI imaging system are the main magnet that polarizes tissue, the shim coils for correcting inhomogeneities in the main magnetic field, the gradient system for localizing the magnetic resonance (MR) signal, and the RF system that excites the tissue and detects the resulting signal. With MRI imaging. different magnetic field strengths can be used. The most common strengths are 0.3 T, 1.5 T and 3 T. The higher the strength, the higher the image quality. For example, a 0.3 T magnetic field strength will result in lower quality imaging then a 1.5 T magnetic field strength.

Currently, there is also no autonomous way of objectively analyzing MRI images, with analysis of such images being reliant on physician training and experience. Moreover, due to technical limitations, diagnostic MRI protocols provide a limited number of slices of a target region, which leaves a physician to piece together anatomical information from available axial, sagittal, and/or coronal scans of the patient anatomy. Existing systems also lack a reliable way to easily and autonomously compare a patient's MRI images against a larger database of MRI images. Such comparison can allow a physician to obtain additional information about the severity of a patient's condition. Existing systems also lack the ability to autonomously compare a patient's MRI images at a present time against past images of that patient. In addition, it is not currently possible to screen a patient's MRI images for spinal cord compression, fracture, tumor, infection, among other conditions. Such limitations make it difficult if not impossible to make treatment recommendations based on patient MRI images that would result in a high degree of confidence in treatment outcome.

With low quality images and lack of reliable and/or reproducible image analysis, existing systems pose a diagnostic challenge for physicians. Such limitations can make it difficult to adequately identify key landmarks and conduct measurements, which may in turn lead to decreased accuracy and efficacy of treatment. The limitations of existing image analysis tools can result in complications with surgical planning, including difficulty with navigating tools and implants to necessary sites. Accordingly, additional systems, devices, and methods for identifying locations of surgical sites (e.g., levels of the spine) may be desirable.

### SUMMARY OF DISCLOSURE

Systems, devices, and methods described herein relate to analysis of anatomical images and identification of anatomical components and/or structures. In some embodiments, systems, devices, and methods described herein relate to identification of levels of a spine and other anatomical components associated with those levels.

In some embodiments, a method includes receiving image data of a set of anatomical components of an anatomical structure, the anatomical structure including a set of levels: receiving segmentation data identifying the set of anatomical components in the image data: implementing a first level identification process to generate a first set of level identification outputs, the first level identification process including determining geometrical parameters of the set of anatomical components based on the segmentation data and grouping the set of anatomical components into separate levels based on geometrical parameters of the set of anatomical components: implementing a second level identification process to generate a second set of level identification outputs, the second level identification process including processing the image data of the set of anatomical components using a machine learning model to generate probability maps for each class of a plurality of classes associated with a set of level types or the set of levels: assigning a level identifier of a level from the set of levels to each anatomical component from the set of anatomical components based on the first and second sets of level identification outputs: and generating a visual representation of the anatomical structure including a visual depiction of the anatomical structure and visual elements indicative of the level identifiers assigned to the set of anatomical components.

In some embodiments, a method includes: receiving a set of two-dimensional (2D) images of a three-dimensional volume containing a set of anatomical components of an anatomical structure, the anatomical structure including a set of levels, the set of 2D images including subsets of 2D images each associated with a different anatomical component from the set of anatomical components: for each anatomical component from the set of anatomical components: processing, using a convolutional neural network (CNN) trained to identify the set of levels or level types of the set of levels, each 2D image from the subset of 2D images associated with the anatomical component to output a predicted level or level type for the anatomical component based on the 2D image: and assigning a level or a level type to the anatomical component based on the predicted levels or level types for the anatomical component output by processing the subset of 2D images associated with the anatomical component: and generating a visual representation of the anatomical structure including a visual depiction of the anatomical structure and visual elements indicative of the level or level type assigned to the set of anatomical components.

### BRIEF DESCRIPTION OF THE DRAWINGS

FIG. 1 is a block diagram illustrating a configuration of a system for collecting and analyzing anatomical images, according to some embodiments.

FIG. 2 is a block diagram illustrating a configuration of a device for level identification of spinal anatomy, according to some embodiments.

FIG. 3 is a schematic illustrating an example model for performing level identification of spinal anatomy, according to some embodiments.

FIG. 4A is a flow chart illustrating a process for training and validating a model for performing level identification of spinal anatomy, according to some embodiments.

FIG. 4B is a flow chart illustrating training of a neural network training process for performing level identification of spinal anatomy, according to some embodiments.

FIG. 5 is a flow chart illustrating a process for level identification of spinal anatomy, according to some embodiments.

FIG. 6A is a flow chart illustrating a process for level identification using anatomical components of vertebrae, according to some embodiments.

FIG. 6B is a flow chart illustrating a process for level identification using intervertebral discs, according to some embodiments.

FIG. 6C is a flow chart illustrating a process for axial image-based level identification, according to some embodiments.

FIG. 6D is a flow chart illustrating a process for sagittal or coronal image-based level identification, according to some embodiments.

FIG. 7 is an example 2D axial scan of a spine of a patient, according to some embodiments.

FIG. 8A is an example 2D axial scan of a spine of a patient with a predicted level type class, according to some embodiments. FIG. 8B is an example of another 2D axial scan of a spine of a patient with a predicted level type class, according to some embodiments.

FIG. 8C is a perspective view of a 3D model of a vertebra, according to some embodiments.

FIG. 9 is an example segmented 3D spine model, according to some embodiments.

FIG. 10A is a perspective view of a 3D model of pedicles with bounding boxes, according to some embodiments. FIG. 10B is a perspective view of a 3D model of pedicles and associated vertebral body with intersecting bounding boxes, according to some embodiments. FIG. 10C is a perspective view of a 3D model of a level of a spine with associated anatomical components and their bounding boxes, according to some embodiments.

FIG. 11 is an example 3D spine model of a patient with levels of the spine identified, according to some embodiments.

FIG. 12A is a 2D radiograph of patient spinal anatomy, according to some embodiments. FIG. 12B depicts a segmentation output or labels associated with the 2D radiograph of FIG. 12A, according to some embodiments. FIG. 12C depicts level identification of the levels of the spine in the 2D radiograph of FIG. 12A, according to some embodiments.

FIG. 13A is an example image of patient anatomy produced using MRI, according to some embodiments. FIG. 13B depicts a segmentation output or labels associated with the MRI image of FIG. 13A, according to some embodiments. FIG. 13C depicts level identification of the levels of the spine in the MRI image of FIG. 13A, according to some embodiments.

FIGS. 14A-14C depict different examples of augmented reality views of patient anatomy, according to some embodiments.

### DETAILED DESCRIPTION

### 1. Overview of System

Systems, devices, and methods described herein relate to processing of patient anatomical structures, including a spine. While certain examples presented herein may generally relate to processing of image data of a spine, it can be appreciated by one of ordinary skill in the art that such systems, devices, and methods can be used to process image data of other portions of patient anatomy, including, for example, vessels, nerves, bone, and other soft and hard tissues near the brain, heart, or other regions of a patient's anatomy.

Systems, devices, and methods described herein can be suited for processing several different types of image data, including X-ray, CT, MRI, fluoroscopic, ultrasound, etc. In some embodiments, such systems, devices, and methods can process a single image type and/or view, while in other embodiments, such systems, devices, and methods can process multiple image types and/or view. In some embodiments, multiple image types and/or views can be combined to provide richer data regarding a patient's anatomy.

Systems, devices, and methods described herein can implement machine learning models to process and/or analyze image data regarding a patient's anatomy. Such machine learning models can be configured to identify and differentiate between different anatomical parts within anatomical structures. In some embodiments, machine learning models described herein can include neural networks, including deep neural networks with multiple layers between input and output layers. For example, one or more convolutional neural networks (CNNs) can be used to process patient image data and produce segmentation outputs that classify different objects within the image data and/or identify different levels of the spine in the image data. Suitable examples of segmentation models and the use thereof are described in U.S. Patent Application Publication No. 2019/0105009, published Nov. 11, 2019, titled “Automated Segmentation Of Three Dimensional Bony Structure Images,” U.S. Patent Application Publication No. 2020/0151507,published May 14, 2020, titled “Autonomous Segmentation Of Three-Dimensional Nervous System Structures From Medical Images,” U.S. Patent Application Publication No. 2020/0410687, published Dec. 31, 2020, titled “Autonomous Multidimensional Segmentation Of Anatomical Structures On Three-Dimensional Medical Imaging,” and U.S. Provisional Patent Application No. 63/187,777, filed May 12, 2021, titled “Systems, Devices, and Methods for Segmentation of Anatomical Image Data,” the disclosures of each of which is incorporated herein by reference. Suitable examples of methods of level identification are described in U.S. Patent Publication No. 2020/0327721, published Oct. 15, 2020, the contents of which are incorporated herein by reference. While certain examples described herein and in such examples employ CNNs, it can be appreciated that other types of machine learning algorithms can be used to process patient image data, including, for example, support vector machines (SVMs), decision trees, k-nearest neighbor, and artificial neural networks (ANNs).

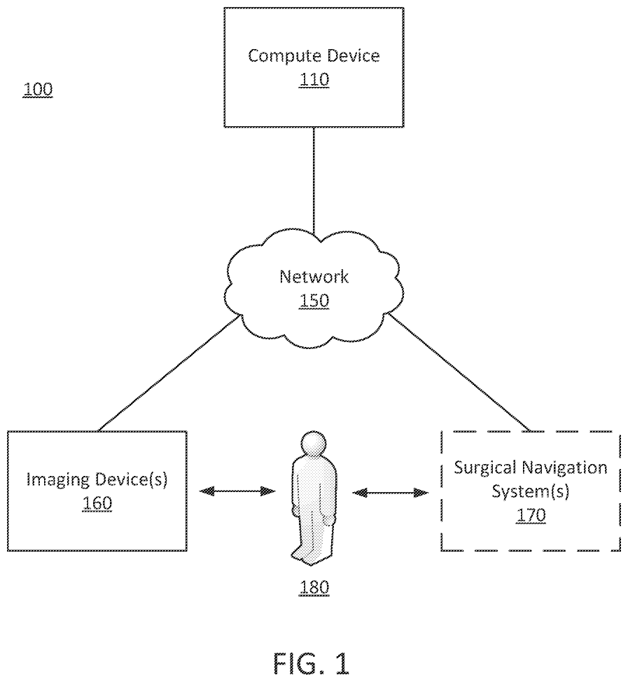

FIG. 1 is a high-level block diagram that illustrates a system 100 for processing image data of patient anatomy and/or providing image guidance to physicians during a surgical procedure, according to some embodiments. The system 100 can include a compute device 110, an imaging device(s) 160, and, optionally, a surgical navigation system(s) 170. In some embodiments, the compute device 110 can communicate with one or more imaging device(s) 160 and optionally one or more surgical navigation system(s) 170, e.g., to perform segmentation of patient anatomical structures, to perform level identification of patient anatomical structures, and/or to provide digital guidance to surgeons during surgical procedures.

In some embodiments, the compute device 110 may be configured to perform segmentation of anatomical image data to identify anatomical parts of interest. For example, the compute device 110 can be configured to generate segmentation outputs that identify different anatomical parts of interest. Additionally, the compute device 110 may be configured to perform level identification of different regions of the spine. The compute device 110 can be configured to generate level identification outputs, such as, for example, a level type (e.g., sacrum, thoracic, lumbar, cervical), a vertebral level (ordinal identifier), or a pair or range of vertebral levels associated with a vertebrae (and/or other nearby anatomical part(s)). Optionally, the compute device 110 can be configured to generate virtual representations of patient anatomy and/or surgical instruments, e.g., to provide image guides to surgeons during surgical procedures. The compute device 110 may be implemented as a single compute device, or be implemented across multiple compute devices that are connected to each other and/or the network 150. For example, the compute device 110 may include one or more compute devices such as servers, desktop computers, laptop computers, portable devices, databases, etc. Different compute device may include component(s) that are remotely situated from other compute devices, located on premises near other compute devices, and/or integrated together with other compute devices.

In some embodiments, the compute device 110 can be located on a server that is remotely situated from one or more imaging device(s) 160 and/or surgical navigation system(s) 170. For example, an imaging device 160 and a surgical navigation system 170 can be located in a surgical operating room with a patient 180, while the compute device 110 can be located at a remote location but be operatively coupled (e.g., via network 150) to the imaging device 160 and the surgical navigation system 170. In some embodiments, the compute device 110 can be integrated into one or both of the imaging device 160 and the surgical navigation system 170. In some embodiments, system 100 includes a single device that includes the functionality of the compute device 110, one or more imaging device(s) 160, and one or more surgical navigation system(s) 170, as further described herein.

In some embodiments, the compute device 110 can be located within a hospital or medical facility. The compute device 110 can be operatively coupled to one or more databases associated with the hospital, e.g., a hospital database for storing patient information, etc. In some embodiments, the compute device 110 can be available to physicians (e.g. surgeons) for performing evaluation of patient anatomical data (including, for example, level data as described herein), visualization of patient anatomical data, diagnoses, and/or planning of surgical procedures. In some embodiments, the compute device 110 can be operatively coupled to one or more other compute devices within a hospital (e.g., a physician workstation), and can send level outputs and/or other image processing outputs to such compute devices (e.g., via network 150) for performing evaluation of patient anatomical data, visualization of patient anatomical data, diagnoses, and/or planning of surgical procedures.

Network 150 may be any type of network (e.g., a local area network (LAN), a wide area network (WAN), a virtual network, a telecommunications network) implemented as a wired network and/or wireless network and used to operatively couple compute devices, including system 100. As shown in FIG. 1, a connection may be defined between compute device 110 and any one of imaging device(s) 160, surgical navigation system(s) 170, and/or other compute devices (e.g., databases, servers, etc.). In some embodiments, the compute device 110 may communicate with imaging device(s) 160 and/or surgical navigation system(s) 170 (e.g., send data to and/or receive data from such devices) and with the network 150 via intermediate networks and/or alternate networks (not shown in FIG. 1). Such intermediate networks and/or alternate networks may be of a same type and/or a different type of network as network 150. Each of the compute device 110, imaging device(s) 160, and surgical navigation system(s) 170 may be any type of device configured to send data over the network 150 to send and/or receive data from one or more of the other devices.

In some embodiments, an imaging device 160 may refer to any device configured to image anatomical structures of a patient 180. In some embodiments, the imaging device 160 may include one or more sensors for measuring signals produced by various imaging technologies. The imaging device 160 can employ a non-invasive technology to image a patient's anatomy. Non-limiting examples of imaging devices include CT scanners, MRI scanners, X-ray devices, ultrasound devices, and combinations thereof, and the like. The image data generated by the imaging device 160 may be transmitted to any of the devices connected to network 150, including, for example, compute device 110. In some embodiments, the image data generated by the imaging device 160 can include a 2D image of an anatomical structure. In some embodiments, the image data generated by the imaging device 160 can include a plurality of 2D image scans that together provide image data for a 3D volume. The imaging device 160 can transmit the image data to the compute device 110 such that the compute device 110 can perform level identification of the patient anatomy and/or label different anatomical parts of interest in the patient anatomy. Optionally, the imaging device 160 can provide the image data to a surgical navigation system 170 such that the surgical navigation system can generate one or more virtual representations of the patient anatomy, e.g., for use in image-guided surgery.

The surgical navigation system 170 can be configured to provide image-guided surgery, e.g., during a surgical operation. For example, the surgical navigation system 170 may facilitate one or more of planning, visualization, and guidance during a surgical procedure. In some embodiments, the surgical navigation system 170 can include a tracking system for tracking patient anatomy, surgical tool(s), implant(s), or other objects within a surgical field. In some embodiments, the surgical navigation system 170 can include an image generator for generating one or more virtual representations of patient anatomy and/or surgical tool(s), implant(s), or other objects within a surgical field and to display these to a physician or other healthcare provider (e.g., a surgeon). In some embodiments, the surgical navigation system 170 can be configured to present a 3D display, e.g., via a 3D wearable device and/or a 3D projector or screen. In some embodiments, the surgical navigation system 170 can be configured to display a position and/or orientation of one or more surgical instrument(s) and implant(s) with respect to presurgical or intraoperative medical image data of the patient anatomy. The image data can be provided, for example, by an imaging device 160, and the surgical navigation system 170 can use the image data to generate a virtual representation of one or more anatomical parts of interest along with position and/or orientation data associated with a surgical device. Suitable examples of surgical navigation systems are described in U.S. Patent Application Publication No. 2019/0053851, published Feb. 21, 2019, and incorporated herein by reference.

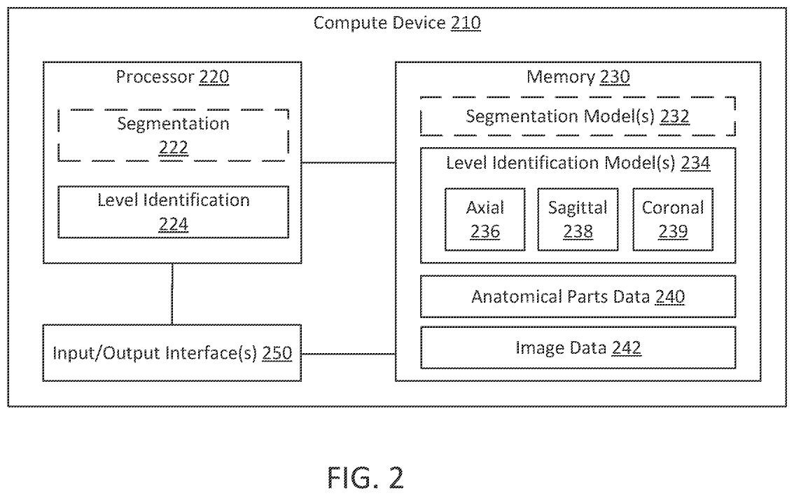

FIG. 2 schematically illustrates an example compute device 210 for surgical planning and/or navigation, according to some embodiments. Compute device 210 can be structurally and/or functionally similar to compute device 110. While a single compute device 210 is schematically depicted, it can be appreciated that the compute device 210 can be implemented as one or more compute devices. In some embodiments, compute device 210 may be configured to identify levels of spinal anatomy of a patient (e.g., patient 180). Compute device 210 includes a processor 220, a memory 230, and one or more input/output interface(s) 250.

Memory 230 may be, for example, a random access memory (RAM), a memory buffer, a hard drive, a database, an erasable programmable read-only memory (EPROM), an electrically erasable read-only memory (EEPROM), a read-only memory (ROM), and/or so forth. In some embodiments, memory 230 stores instructions that cause processor 220 to execute modules, processes, and/or functions associated with segmentation 222 and level identification 224. Memory 230 can store one or more segmentation models 232, level identification model(s) 234, anatomical parts data 240, and/or image data 242.

The segmentation models 232 can be models or algorithms for performing image-based segmentation, whereby different portions of anatomical image data can be classified or labeled. In some embodiments, the segmentation models 232 can include machine learning models, such as, for example, a CNN model, a SVM model, etc. The segmentation models 232 can be implemented by the processor 220 to perform segmentation 222. In some embodiments, the segmentation models 232 can be unique to particular anatomical regions, e.g., spinal anatomy, cardiac anatomy, etc. In some embodiments, the segmentation models 232 can be unique to particular image types, e.g., X-ray, CT, MRI, etc.

The level identification models 234 can be models or algorithms for identifying and/or labeling different levels of the vertebrae of the spine and/or other anatomical parts associated with those levels (e.g., nerves, intervertebral discs, etc.). In some embodiments, the level identification models 234 can include machine learning models, such as, for example, a CNN model, a SVM model, etc. The level identification models 234 can be implemented by the processor 220 to perform level identification 224. In some embodiments, the level identification models 234 can be unique to particular image types (e.g., X-ray, CT, MRI) and/or image views. For example, the level identification models 234 can include an axial model 236 for identifying levels in axial image data, a sagittal model 238 for identifying levels in sagittal image data, and/or a coronal model 239 for identifying levels in sagittal image data.

The anatomical parts data 240 can include information relating to anatomical parts of a patient. For example, the anatomical parts data 240 can include information identifying, characterizing, and/or quantifying different features of one or more anatomical part(s), such as, for example, a location, color, shape, geometry, or other aspect of an anatomical part. The anatomical parts data 240 can enable processor 220 to perform segmentation 222 and/or anatomical parts identification 224 based on patient image data. The image data 242 can include image data associated with one or more patient(s) and/or information about different image devices, e.g., different settings of different image devices (e.g., image device(s) 160) and how those settings may impact images captured using those devices.

The processor 220 may be any suitable processing device configured to run and/or execute any of the functions described herein. In some embodiments, processor 220 may be a general purpose processor, a Field Programmable Gate Array (FPGA), an Application Specific Integrated Circuit (ASIC), a Digital Signal Processor (DSP), a Dedicated Graphics Processing Unit (GPU), and/or the like. In some embodiments, the processor 220 can be configured to perform one or more of segmentation 222 and level identification 224. Segmentation 222 and level identification 224 can be implemented as one or more programs and/or applications that are tied to hardware components (e.g., processor 220, memory 230, input/output interface(s) 250). In some embodiments, a system bus (not shown) may be configured to enable processor 220, memory 230, input/output interface(s) 250, and/or other components of the compute device 210 to communicate with each other.

The input/output interface(s) 250 may include one or more components that are configured to receive inputs and send outputs to other devices (e.g., imaging device(s) 160, surgical navigation system(s) 170, etc.). In some embodiments, the input/output interface(s) 250 can include a user interface, which can include one or more components that are configured to receive input and/or present output to a user. For example, input/output interface 250 may include a display device (e.g., a display, a touch screen, etc.), an audio device (e.g., a microphone, a speaker), a keypad, and/or other interfaces for receiving information from and/or presenting information to users. In some embodiments, the input/output interface 250 can include a communications interface for communicating with other devices, and can include conventional electronics for data communication using a standard communication protocol, e.g., Wi-Fi, Bluetooth®, etc.

### 2. Overview of Level Identification Methods

Systems, devices, and methods described herein can identify levels of spinal anatomy, e.g., different level types and/or ordinal identifiers. As described above, a compute device (e.g., compute devices 110, 210) for performing segmentation and/or level identification can implement one or more algorithms or models. In some embodiments, the algorithms or models can include machine learning models, which can be trained using labeled training datasets. The machine learning models can use the training datasets to learn relationships between different features in the image data and the output labels.

In some embodiments, systems, devices, and methods described herein can perform pre-processing of image data prior to performing segmentation and/or level identification. In many instances, image data collected using conventional imaging techniques can have low quality. For example, to avoid the risks of exposing patients to high levels of radiation, a CT imaging device may be used on a lower dose setting to capture images of patient anatomy. Similarly, MRI imaging devices using lower power may be used to capture images of patient anatomy. Such low dose or low power images can have images that have a higher amount of noise. A compute device (e.g., compute devices 110, 210) as described herein can optionally pre-process the image to remove such noise prior to performing segmentation and/or spinal level identification.

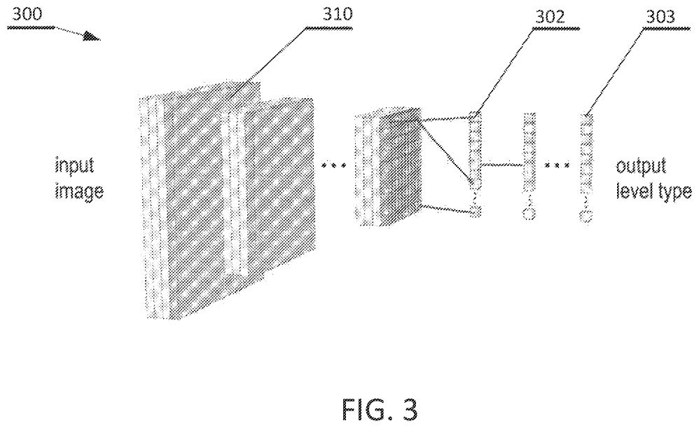

FIG. 3 is a schematic illustrating a CNN model 300 for performing level identification of spinal anatomy, according to embodiments. The CNN model 300 can be an example of a level identification model, such as, for example, level identification model 234 described with reference to FIG. 2. The CNN model 300 may be configured to perform vertebral level prediction on image data, including, for example, 2D scans (e.g., DICOM slices) of anatomical structures. In some embodiments, the CNN 300 can be configured to return one or more probability maps that identify for each vertebral level class (e.g., thoracic, sacrum, lumbar, cervical), a probability that an anatomical image (or portion of the image, e.g., pixel or group of pixels) belongs to that particular class. The probabilities associated with the different classes can sum to one, such that a class having the largest probability is indicative of the most likely class to which the image (or portion of the image) belongs.

In some embodiments, the input to the CNN model 300 may be a contracting path (encoder) and includes a plurality of stacked convolution blocks 310 including one or more convolution layers and/or pooling layers. For example, each convolution block can include two convolutional layers with an optional batch normalization layer between them, and followed with a pooling layer. One or more images (e.g., raw images or denoised images) can be presented to the input layer of the CNN model 300, and the CNN model 300 via the series of convolution layers and/or pooling layers can extract features from the image data. The image data can include a single image (e.g., an X-ray image or a single image scan) or a set of images of 2D scans that together form a local volume representation. In some embodiments, the convolution layers can be of a standard kind, the dilated kind, or a combination thereof, with ReLU or leaky ReLU activation attached.

In some embodiments, the last convolution block 310 may be directly connected to a plurality of dense, fully-connected layers 302 that are stacked together. In some embodiments, each fully-connected layer 302 may be preceded by a dropout layer, and each fully-connected layer may optionally have a ReLU or leaky ReLU activation function attached. The last fully-connected layer 303 may be considered a network output layer that corresponds to all possible outputs. For example, the possible outputs can include all vertebral type classes (e.g., cervical, thoracic, lumbar, sacrum). In some embodiments, output layer 303 generates a probability map for each output class (e.g., vertebral type class) with a Softmax or Sigmoid activation function for converting output scores into a normalized probability distribution.

The CNN model 300 can be configured to process images of different sizes by adjusting the size (e.g., resolution) of the layers. Depending on requirements of particular applications, one or more of the number of layers, the number of filters within a layer, the dropout rate for dropout layers, etc. can be adjusted. For example, deeper networks with a greater number of layers and/or filters can give results with better quality, but increasing the number of layers and/or filters can significantly increase the computation time and decrease the capability of the CNN model 350 to generalize. Therefore, a greater number of layers and/or filters can be impractical for certain applications. In some embodiments, the CNN model 300 can be supplemented with additional skipping connections of layers with corresponding sizes (e.g., resolutions), which can improve performance through information merging.

As described above, the CNN model 300 can be used to perform level identification of spinal anatomy. For example, the CNN model 300 can be configured to classify portions of images (e.g., each voxel/pixel or groupings of voxel/pixels) into different level type classes, e.g., sacrum and/or cervical, thoracic, and/or lumbar spine. In some embodiments, the CNN model 300 can be configured to classify portions of images into different vertebral level (ordinal identifier) classes, e.g., thoracic levels 1-12 (T1-T12), lumbar levels 1-5 (L1-L5), sacral levels 1-5 (S1-S5), and/or cervical levels 1-8 (C1-C8). In some embodiments, a first CNN model can be configured to perform a first classification (e.g., vertebral level type), and the output of that first CNN model can be combined and inputted into one or more additional CNN models that are configured to perform one or more additional classifications (e.g., ordinal identifier). In some embodiments, the CNN model 300 can be configured to classify images by identifying a pair of spine levels (e.g., L1/L2, C6/C7, etc.) or a range of spine levels (e.g., C5-T7, L1-L4, etc.). As described above, the CNN model 300 can be trained to identify patient anatomy using a training dataset including images with labeled anatomical parts.

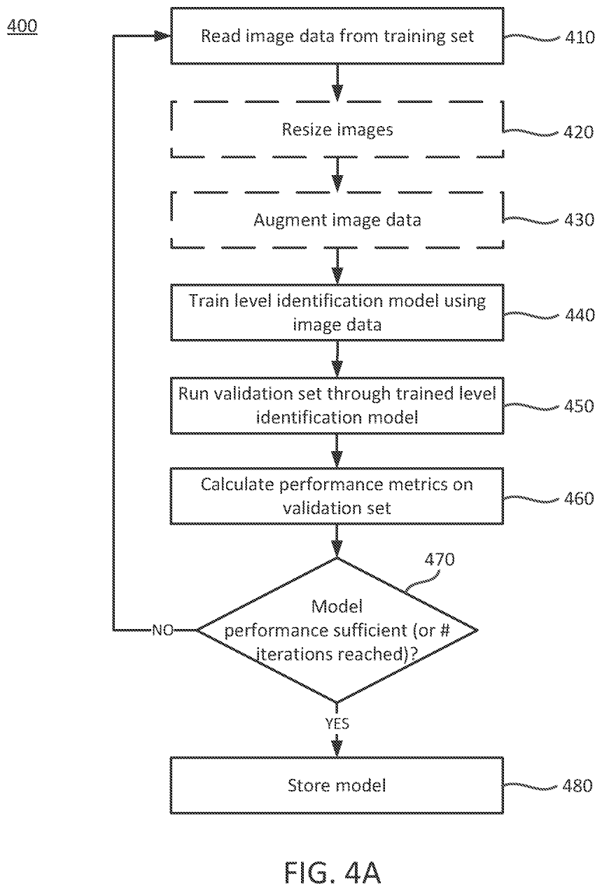

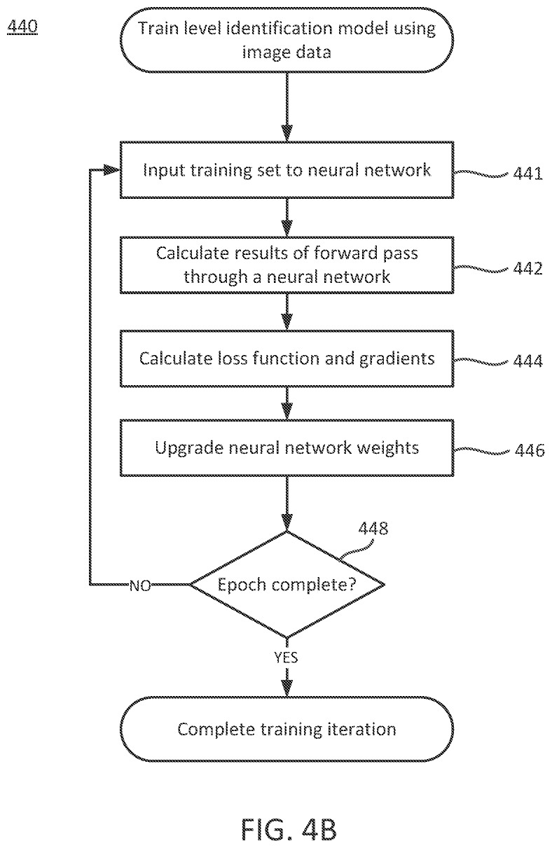

Further details of the training and use of CNN models are discussed with reference to the flow diagrams depicted in FIGS. 4A-6D. The methods depicted in FIGS. 4A-6D can be implemented by one or more devices as described with reference to FIGS. 1 and 2, including, for example, compute device 110, 210.

### 3. Level Identification Model Training

FIG. 4A is a flow chart of a method 400 of training a level identification model (e.g., CNN model 300). The method 400 may include reading image data from a training dataset, at 410. The training dataset can include input images of anatomical structures (e.g., spine, nerves, intervertebral discs, etc.) and corresponding output images of anatomical structures with labelling applied to different parts of the anatomical structures. The images can be grouped into multiple batches for training the level identification model. Each image within a batch can include images representative of a series of slices of a 3D volume of an anatomical structure. Each output image can include at least one label which identifies a portion of that image as corresponding to a vertebral level type, vertebral level, or a pair or range of vertebral levels. In some embodiments, each output image can include one or more labels, with the labels indicating different vertebral level types and/or vertebral levels. A compute device (e.g., compute device 110, 210) can read the image data by loading one or more batches of images for further processing.

Optionally, the images read from the training dataset can be resized, at 420. For example, the images captured by different imaging devices can vary in size, and therefore a base size can be established for inputting into the level identification model. Images that do not conform to the base size can be resized, e.g., using a resizing function.

Optionally, at 430, the image data may be augmented. Data augmentation can be performed on the image data to create a more diverse set of images. Each input image and its corresponding output image can be subjected to the same data augmentation, and the resulting input and output images can be stored as new images within the training dataset. The data augmentation can include applying one or more transformations or other data processing techniques to the images. These transformations or processing techniques can include: rotation, scaling, movement, horizontal flip, additive noise of Gaussian and/or Poisson distribution and Gaussian blur, etc. Data augmentation can be performed on any image type, including, for example, X-ray, CT scans, and/or MRI scans, as well as any image view (e.g., axial, sagittal, coronal). In some embodiments, data augmentation can be performed on 3D image data (e.g., 3D CT image data including 2D scans of a 3D volume), and the augmented 3D image data can be used to construct 2D images.

At 440, a level identification model may be trained using the training dataset, including the original image data and/or the augmented image data. In some embodiments, the training can be supervised. The training can include inputting the input images into the level identification model, and minimizing differences between an output of the level identification model and the output images (including labeling) corresponding to the input images. In some embodiments, the level identification model can be a CNN model, whereby one or more weights of a function can be adjusted to better approximate a relationship between the input images and the output images. Further details of training a CNN model are described with reference to FIG. 4B. In some embodiments, the training can be unsupervised, e.g., where the level identification model relies on a distance between feature vectors to classify unknown data points.

A validation dataset may be used to assess one or more performance metrics of the trained level identification model. Similar to the training dataset, the validation dataset can include input images of anatomical structures (e.g., spine, nerves, intervertebral discs, etc.) and output images including labelled parts of the anatomical structures. The labels can be, for example, different vertebral level type(s), vertebral level(s), and/or a pair or range of vertebral levels associated with the different parts. The validation dataset can be used to check whether the trained level identification model has met certain performance metrics or whether further training of the level identification model may be necessary. At 450, input images of a validation dataset can run through the trained level identification model to obtain outputs. At 460, one or more performance metrics can be calculated based on the outputs of processing the validation dataset. For example, the outputs of the validation dataset can be compared to the output images that correspond to the input images, and differences between the outputs of the model and the output images can be evaluated on a qualitative and/or quantitative scale. Different performance metrics can be calculated based on the differences between the outputs of the model and the output images corresponding to the input images. For example, a number or percentage of pixels (or groupings of pixels) that are classified correctly or incorrectly can be determined, and/or a Sorensen-Dice coefficient may be calculated.

At 470, the compute device can determine whether training is completed (e.g., performance of the trained level identification model is sufficient and/or a certain number of training iterations has been met) or whether further training is necessary. In some embodiments, the compute device can continue to cycle through training iterations (i.e., proceed back to 410-460) until the performance of the trained model no longer improves by a predetermined amount (i.e., the performance metrics of a later training iteration 410-460 do not differ from the performance metrics of an earlier training iteration 410-460 by a predefined threshold value or percentage). If the model is not improving, the level identification model may be overfitting the training data. In some embodiments, the compute device can continue to cycle through training iterations (i.e., proceed back to 410-460) until the performance metrics of a training iteration 410-460 reaches a certain predefined threshold indicative of sufficient performance. In some embodiments, the compute device can continue to cycle through training iterations (i.e., proceed back to 410-460) until a predefined number of iterations has been met (i.e., the level identification model has been trained a predefined number of times).

Once the level identification model has been sufficiently trained (470: YES), the level identification model can be stored, e.g., in a memory (e.g., memory 230), at 480. The stored level identification model can be used by the compute device in an inference or prediction process, e.g., to perform level identification on new image data of a patient.

FIG. 4B is a flow chart of training of a level identification model (440 of method 400) implemented as a neural network such as, for example, a CNN (e.g., CNN model 300). The neural network model can be trained by adjusting or tuning the parameters of the neural network model to be able to classify different portions of the image data based on features extracted from those portions. The neural network after being trained can be used to perform level identification (e.g., level type prediction, vertebral level (ordinal identifier) prediction, etc.) on a plurality of images (e.g., 2D scans of patient anatomy) and/or to combine the outputs of the level identification into a 3D model that provides level identification information of the spine and/or other neighboring anatomical structure(s).

The method 440 can include inputting a batch of image data from a training dataset to a neural network, at 441. As described above, the training dataset can include input images of patient anatomy and corresponding output images of labeled patient anatomy (e.g., anatomical components such as vertebrae being labeled with level types and/or levels (ordinal identifiers)). Batches of images can be read from the training dataset one at a time, and processed using the neural network. In some embodiments, the batches of images can include augmented images, as described above. For example, certain input and output images can be subjected to one or more transformations or other augmentation techniques, and the transformed or augmented images can be included in a training dataset for training the neural network.

The batch of images can be passed through the layers of the neural network in a standard forward pass, at 442. The forward pass can return outputs or results, which can be used to calculate a value of a loss function, at 444. The loss function or objective function represents the function that is used to evaluate a difference between the desired output (as reflected in the output images that correspond to the input images) and the output of the neural network. The value of the loss function can indicate a measure of that difference between the desired output and the output of the neural network. In some embodiments, the difference can be expressed using a similarity metric, including, for example, a mean squared error, mean average error, or categorical cross-entropy. The value of the loss function can be used to calculate the error gradients, which in turn can be used to update one or more weights or parameters of the neural network, at 446. The weights and parameters, can be updated to reduce the value of the loss function in a subsequent pass through the neural network.

At 448, the compute device can determine whether the training has cycled through the full training dataset, i.e., whether the epoch is complete. If the epoch has been completed, then the process can continue to 450, where a validation dataset is used to evaluate the performance metrics of the trained level identification model. Otherwise, the process may return to 441, where a next batch of images is passed to the neural network.

### 4. Level Identification Prediction

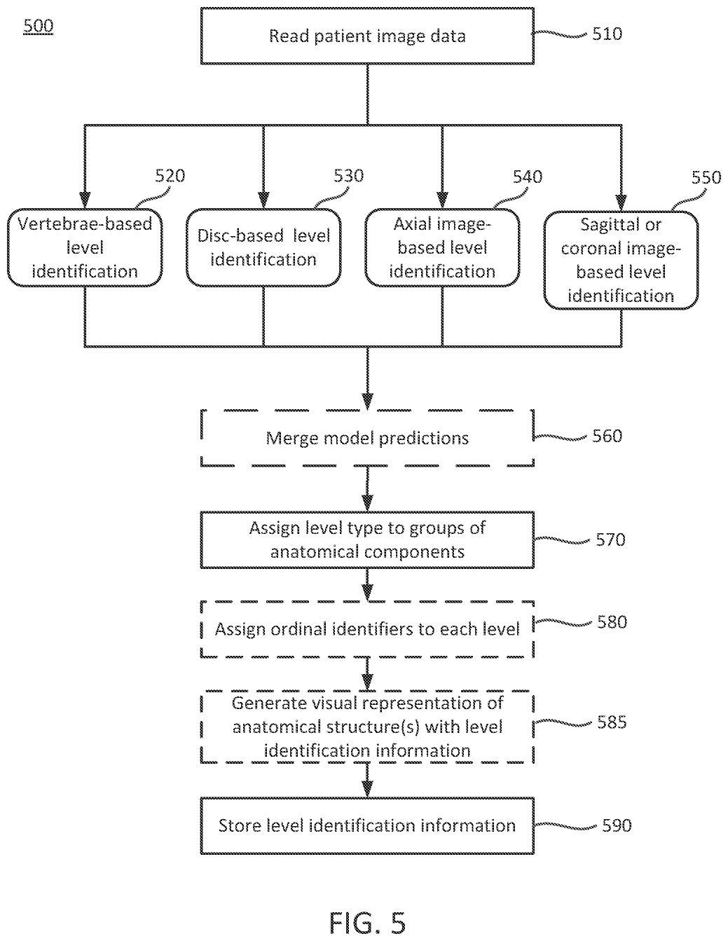

FIG. 5 is a flow chart of a method 500 of performing level identification, e.g., using one or more level identification algorithms or models, according to embodiments. The level identification algorithms or models can include trained level identification models, e.g., as trained per method 400 described with reference to FIGS. 4A and 4B. The method 500 may include reading a batch of images from patient image data, at 510. The images can be new images that are acquired of a patient's anatomical structure. The images can be, for example, 2D scans of a 3D volume of the anatomical structure. In some embodiments, the images can include CT images, MRI images, and/or X-ray images. In some embodiments, the images can include axial, sagittal, and/or coronal views. In some embodiments, the images can be preprocessed. For example, the one or more images can be denoised using a model for denoising image data. Alternatively or additionally, the images can be processed using other techniques, such as, for example, filtering, smoothing, cropping, normalizing, resizing, etc. In some embodiments, the images can be preprocessed such that they share similar parameters to those images that were used to train a level identification model (e.g., resized to have the same size as the images that were used to train the level identification model), as described above with reference to FIGS. 4A and 4B. In some embodiments, inference-time distortions can be applied to one or more images, with a predefined number of distorted images being created for each input image. These distorted images can create inference results that are robust as to small variations in brightness, contract, orientation, etc.

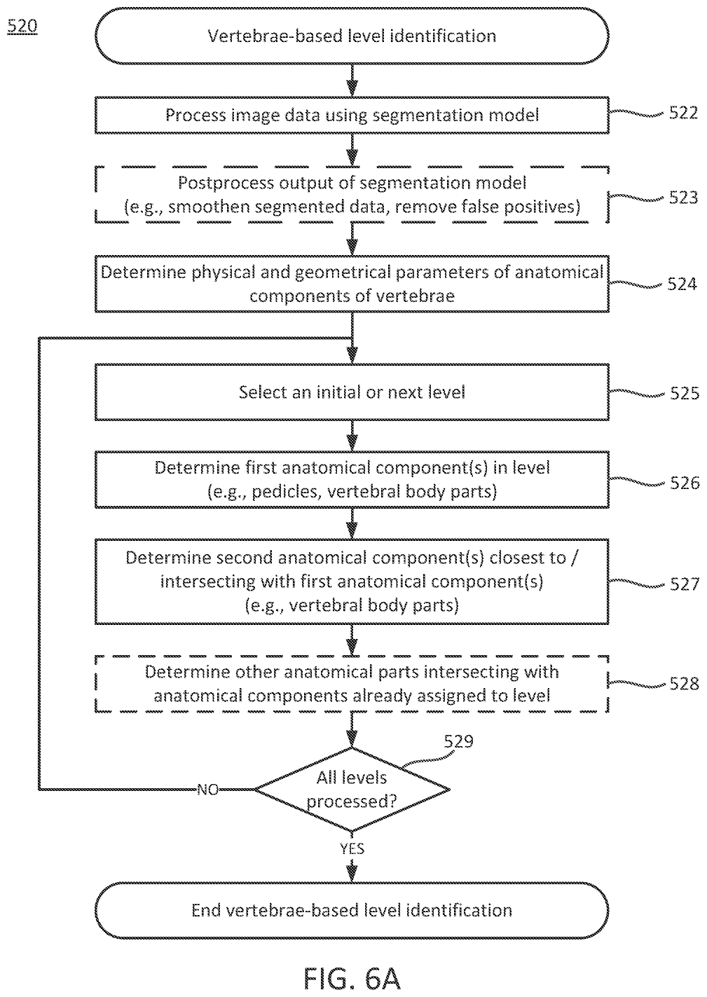

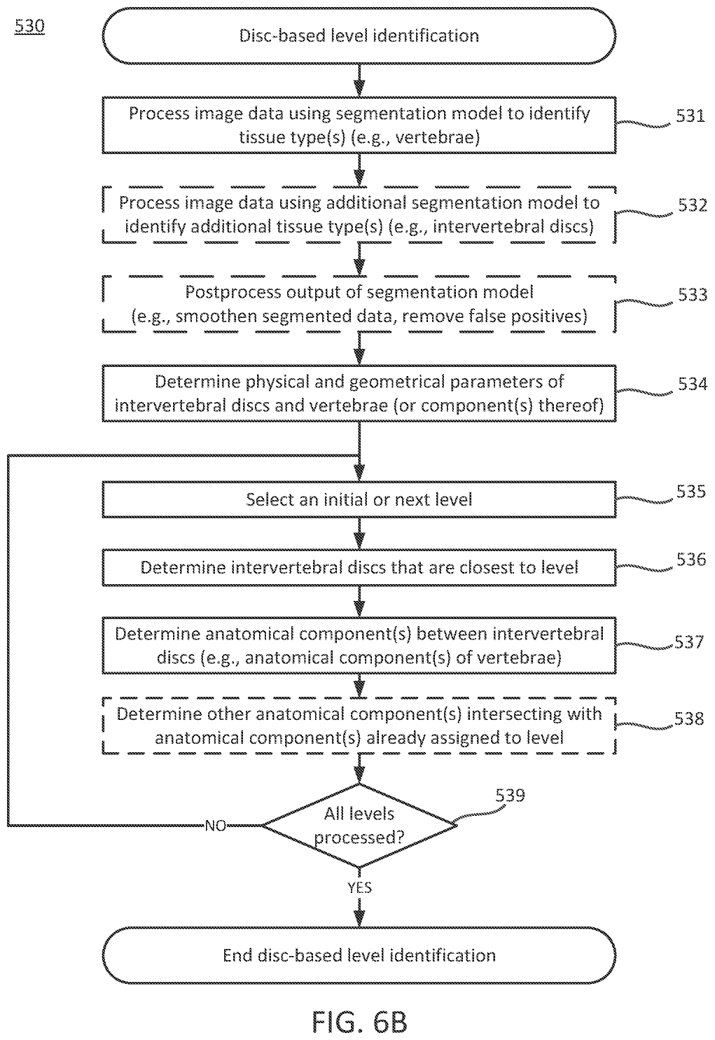

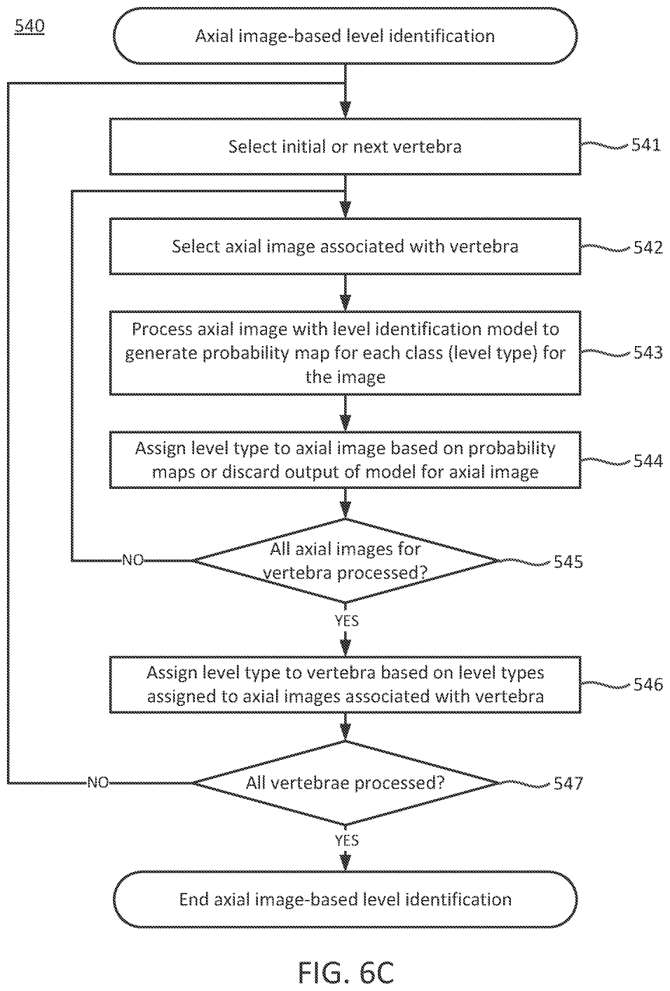

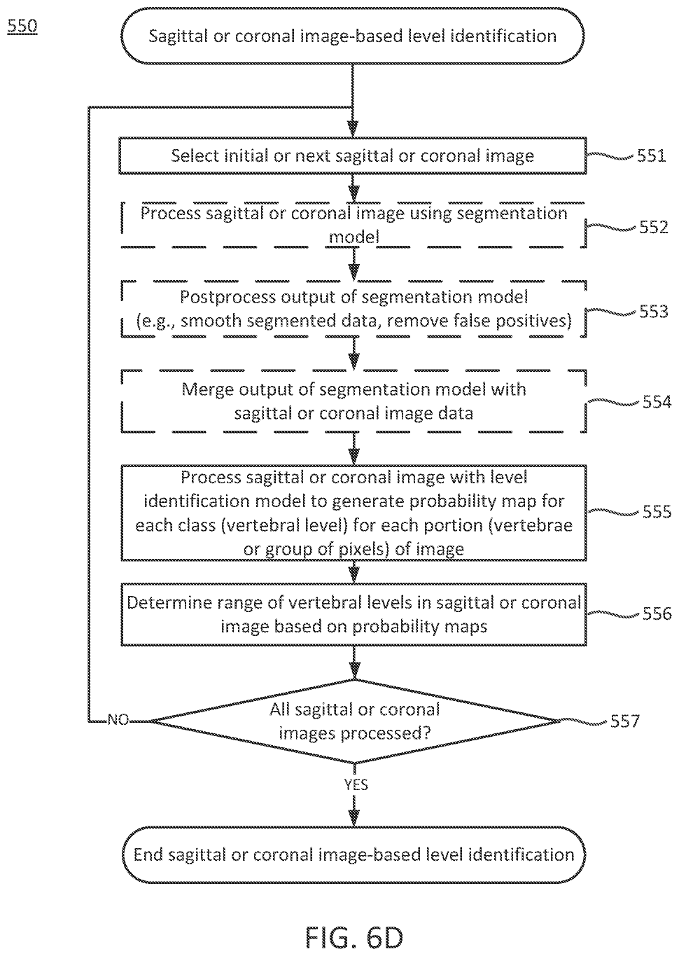

The patient image data may be processed via one or more level identification methods or algorithms, at 520-550. At 520, the image data (or a portion of the image data) may be input to a vertebrae-based level identification process, as described in more detail herein with reference to FIG. 6A. At 530, the image data (or a portion of the image data) may be input to a disc-based level identification process, as described in more detail herein with respect to FIG. 6B. At 540, the image data (or a portion of the image data) may be input to an axial image-based level identification process, as described in more detail herein with respect to FIG. 6C. At 550, the image data (or a portion of the image data) may be input to a sagittal or coronal image-based level identification process, as described in more detail herein with respect to FIG. 6D. In some embodiments, multiple sets of image data (e.g., multiple sets of 2D scans of patient anatomy) can be read, at 510, and processed using different ones of vertebrae-based level identification 520, disc-based level identification 530, axial image-based level identification 540, and/or sagittal or coronal image-based level identification. For example, axial 2D images can be processed using one or more of vertebrae-based level identification 520, disc-based level identification 530, and/or axial image-based level identification 540 but not sagittal or coronal image-based level identification 550. Alternatively, sagittal or coronal 2D images can be processed using one or more of vertebrae-based level identification 520, disc-based level identification 530, and/or sagittal or coronal image-based level identification 550 but not axial image-based level identification 540. As such, depending on the characteristics of the image sets, different processes can be used to generate level identification predictions for those image sets.

Optionally, at 560, if multiple processes 520-550 were used, the level identification predictions from those processes 520-550 can be merged, e.g., according to predetermined schemes (e.g., averaging with or without weighting factors, majority rules, predetermined thresholds, etc.). In some embodiments, different ones of vertebrae-based level identification 520, disc-based level identification 530, axial image-based level identification 540, and/or sagittal or coronal image-based level identification 550 can provide different outputs, which can be used to supplement one another. For example, the outputs of a first process 520-550 can include morphological or spatial relationships of different anatomical parts (e.g., different parts of the vertebrae), while the outputs of a second process 520-550 can include predicted vertebral level types, and the two processes 520-550 can be used to provide more comprehensive data for assigning level types and/or levels (ordinal identifiers) to the vertebrae and/or neighboring anatomical structures.

At 570, for each sub-volume (e.g., voxel/pixel or group of voxels/pixels, or group of anatomical components) of a patient's 3D image data, a level type from the plurality of level types (e.g., cervical (C), thoracic (T), lumbar (L), and/or sacrum(S)) can be assigned based on the predictions of the processes 520-550. When different processes 520-550 operate on the image data differently and therefore produce different level identification outputs, the outputs can be used to supplement one another in assigning level types to the sub-volumes. When different processes 520-550 produce outputs that are the same or substantially similar, the most frequent prediction or highest probability prediction of the vertebral level type for a given sub-volume can be taken into consideration in assigning a level type to that sub-volume. In an example implementation, a level type (e.g., C. T. L or S) can be assigned to each group of anatomical components by combining morphological and spatial relationships determined using vertebrae-based level identification 520 and/or disc-based level identification 530, predictions of vertebral level types and/or vertebral levels determined using axial image-based level identification 540, and/or predictions of vertebral levels or ranges of vertebral levels determined using sagittal or coronal image-based level identification 550. Further details of such outputs are described below with reference to FIGS. 6A-6D.

Optionally, at 580, a vertebral level or ordinal identifier (e.g., C1-S5, or C1-C7, T1-T12,L1-L5 (L6), and/or S1-S5) may be assigned to one or more sub-volumes or groups of anatomical components. Based on the orientation of the patient anatomy in the image data (e.g., 3D volumetric data), level identification outputs, overall distribution of level types, etc., vertebral levels (ordinal identifiers) can be assigned to the levels. In some embodiments, indices can be assigned and counting can be used to assign the ordinal identifiers. For example, counting of lumbar vertebrae may start from L5 (or L6) if the sacrum is included in the image data or from L1 if the thoracic spine is included in the image data. Similar counting can be employed for each of the other vertebrae (e.g., cervical, sacrum, and thoracic). An ordinal identifier can be assigned at 580 to each group of anatomical components belonging to a level type (e.g., C, T, L, S) and based on the anatomical structure and distribution of all the other levels.

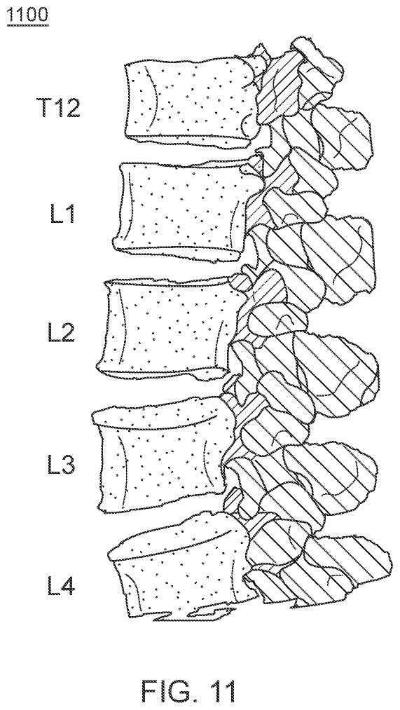

Optionally, one or more virtual representations of the patient anatomy can be generated, at 585, e.g., for visualization in pre-operative planning (e.g., via compute device 110, 210) and/or image-guided surgery (e.g., via a surgical navigation system 170). In some embodiments, a 3D anatomical model may be generated based on the image data and level identification output, which can be used to generate virtual representations of the patient anatomy for visualization. In some embodiments, the 3D anatomical model can be converted or used to generate a polygonal mesh representation of the patient's anatomy (or portion thereof). The parameters of the virtual representation (e.g., volume and/or mesh representation) can be adjusted in terms of color, opacity, mesh decimation, etc. to provide different views of the patient anatomical structure to a user (e.g., a surgeon). In some embodiments, the virtual representations can be 2D views, e.g., a sagittal or coronal view of the patient's anatomy, with labelled vertebral level types or levels. In some embodiments, the virtual representation can be 3D views, e.g., a 3D construction or model of the patient's spine and/or neighboring anatomy (e.g., nerves, discs, etc.). For example, FIG. 11 is a perspective view of a 3D model 1100 of a spine of a patient, according to embodiments. The 3D model 1100 includes a set of assigned ordinal identifiers (i.e., T12, L1, L2, L3, L4) for identifying the different levels of the spine. In some embodiments, the assigned vertebral level type or ordinal identifiers can be depicted using text (e.g., on the side of the relevant vertebrae, or overlaid on the relevant vertebrae) or other visual elements indicative of the vertebral level type or ordinal identifiers (e.g., different coloring, different shading, etc. with a suitable legend). The virtual representations of the patient's anatomy may be visualized, e.g., on a display system of a compute device (e.g., compute device 110, 210) and/or surgical navigation system (e.g., surgical navigation system 170).

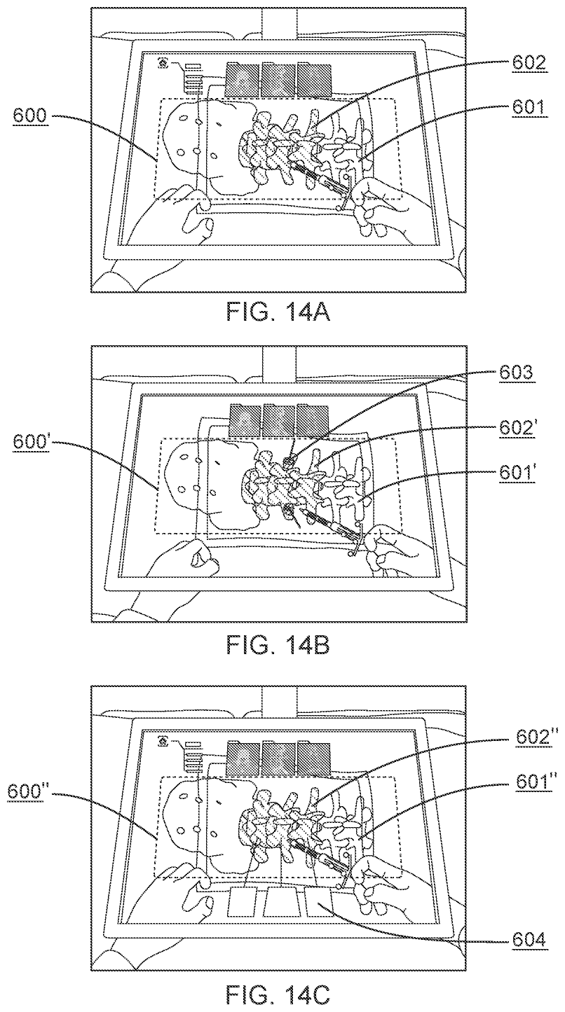

In some embodiments, level identification information can be used to display different configurations of identification levels and/or other components of patient anatomy (e.g., nerves, intervertebral discs, etc.) or surgical instruments (e.g., implants, surgical tools). For example, level identification information can be used to selectively display and hide different levels of the spinal anatomy (and/or nearby structures). Such selective display of anatomical components and/or surgical instruments can allow a surgeon to focus on information that is necessary for a surgical procedure or at specific periods of time during a surgical procedure. In some embodiments, anatomical models of the patient anatomy can be used to provide a virtual or augmented reality image for display by a computer-assisted surgical system, such as, for example, surgical navigation system(s) 170. In such systems, a virtual 2D or 3D view of the patient's anatomy can be displayed over a real portion of the patient anatomy (e.g., a surgical site), wherein level identification information can be used to selectively display different levels of the patient's anatomy, to label different levels of the patient's anatomy, and/or to identify information about specific surgical sites (e.g., an intervertebral disc space between two levels, placement of a screw or other implant into a specific vertebral level, etc.). FIGS. 14A-14C depict examples of augmented reality views of a display of a surgical navigation system, according to embodiments. The augmented reality views depicted in FIGS. 14A-14C can be displayed, for example, by a display device of surgical navigation system 170, as described with reference to FIG. 1. FIG. 14A depicts an augmented reality view 600 with a first set of levels 601 being hidden and a second set of levels 602 being displayed, e.g., to selectively display the levels of interest during a surgical procedure. FIG. 14B depicts an augmented reality view 600′ with hidden levels 601′ and displayed levels 602′ along with an implant 603 that is visible in the region with the displayed levels 602″. In some configurations, the implant can be displayed in a different color or shading, e.g., to emphasize that it is a different material or component from the vertebrae. FIG. 14C depicts an augmented reality view 600″ with hidden levels 601″ and displayed levels 602″ along with one or more label(s) 604 that identify the different levels and/or level types. While the labels 604 are shown as boxes in FIG. 14C, it can be appreciated that the labels 604 can include information regarding vertebral level types, vertebral levels, and/or information (e.g., geometrical characteristics or other features) associated with such levels.

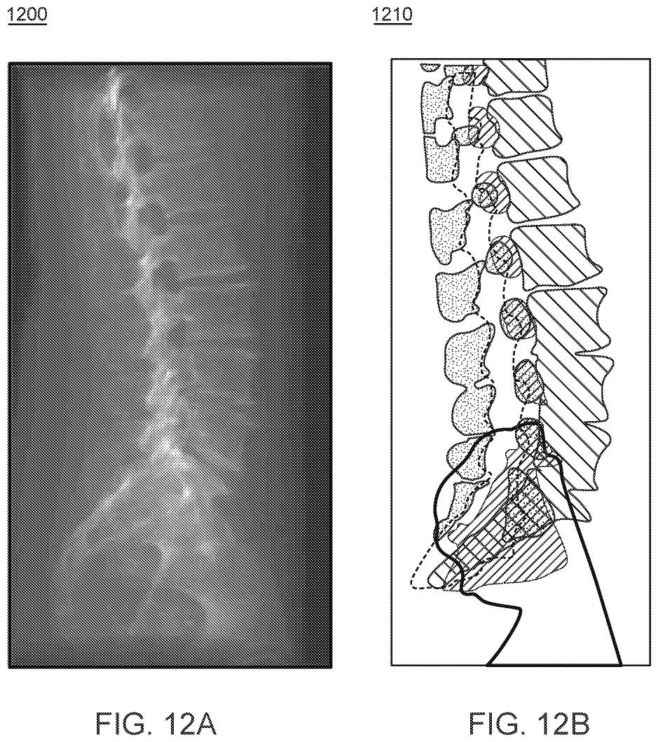

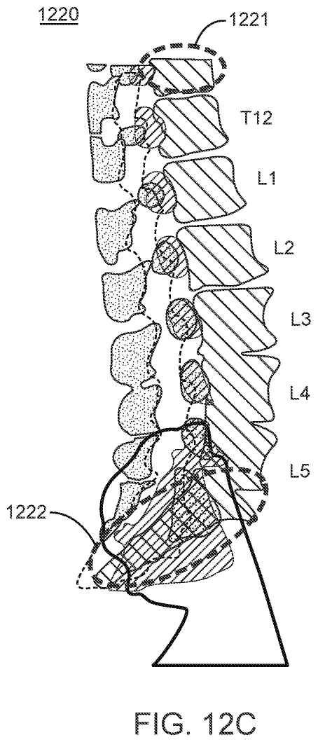

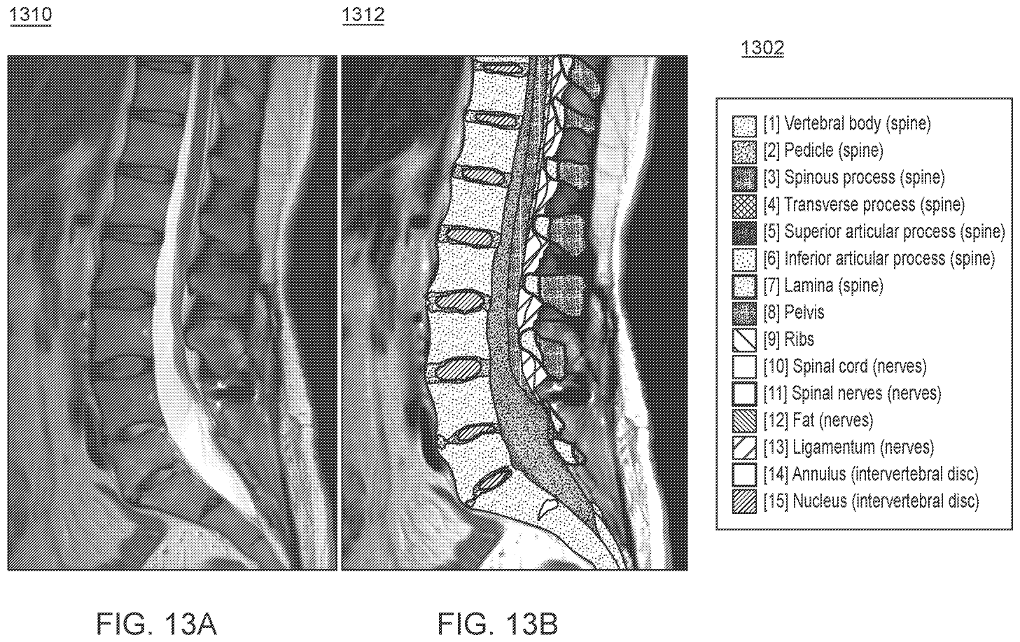

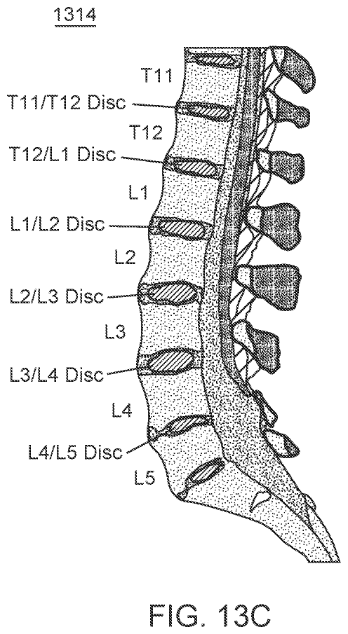

In some embodiments, level identification prediction data can be combined with segmentation output data, e.g., to provide greater depth of data for further analysis and/or display. In some embodiments, segmentation data can be combined with level identification data to enable level identification of anatomical components in addition to the vertebrae of the spine. For example, level identification predictions can be generated for intervertebral discs, nerve roots, etc. FIG. 12C depicts segmentation data produced from processing a X-ray of a patient's spine 1220 together with a set of vertebral levels (ordinal identifiers) assigned to the different levels of the spine (e.g., T12, L1, L2, L3, L4, L5). FIG. 13C depicts segmentation data produced from processing a MRI scan of a patient's spine 1314 together with ordinal identifiers assigned to vertebrae of the spine (e.g., T11, T12, L1, L2, L3, L4, L5) and the intervertebral discs between the vertebrae (e.g., T11/T12 Disc, T12/L1 Disc, L1/L2 Disc, L2/L3 Disc, L3/L4 Disc, L4/L5 Disc). The segmentation output data can be produced, for example, by processing the image data (e.g., one or more X-ray or MRI images) using one or more segmentation models (e.g., segmentation model(s) 232). The ordinal identifiers can be assigned based on the output(s) generated by one or more level identification models (e.g., level identification model(s) 234), as detailed in example methods depicted in FIGS. 5-6D.

In some embodiments, different portions of image data (e.g., a portion of an image) may not produce conclusive level identification predictions and therefore no level identification prediction may be provided for these portions. For example, level identification predictions may not be provided for portions of image data that are cropped (e.g., a partial view of a vertebra) or portions of an image data that are noisy or have lower resolution (e.g., have overlapping vertebral components). FIG. 12C depicts a first region 1221, where a portion of a vertebrae is cropped, and a second region 1222, where multiple vertebrae are shown joined or merged together). As shown in FIG. 12C, level identification predictions were not provided for these regions 1221, 1222.

At 590, the assigned level identification information and/or the outputs of the level identification model can be stored in memory (e.g., memory 230).

### i. Vertebrae-Based Level Identification

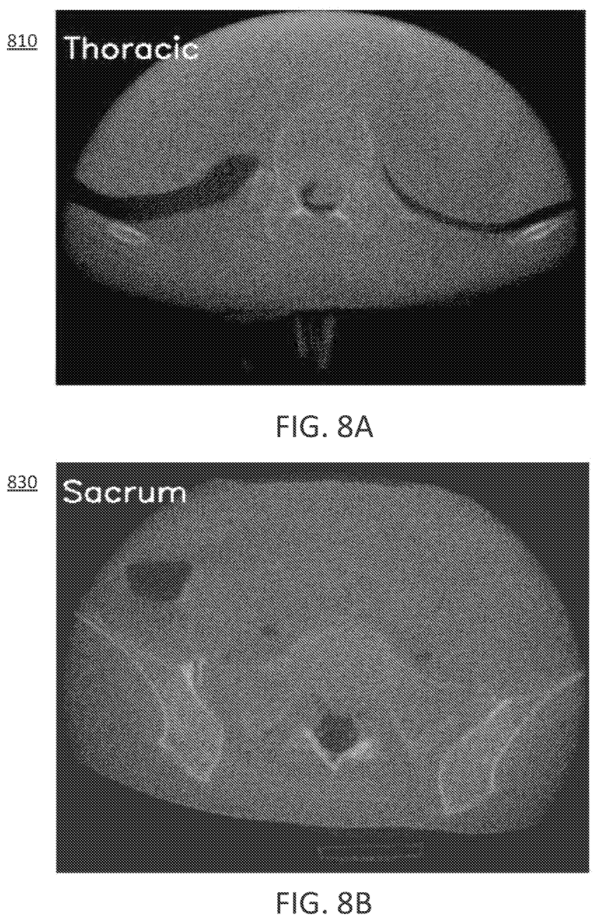

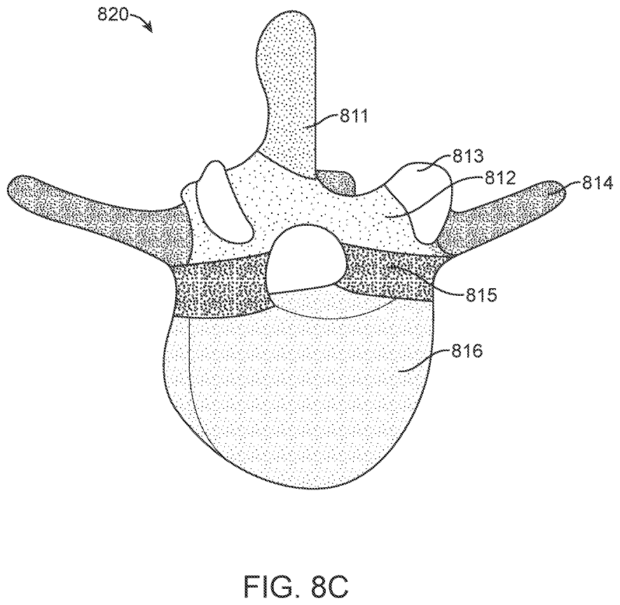

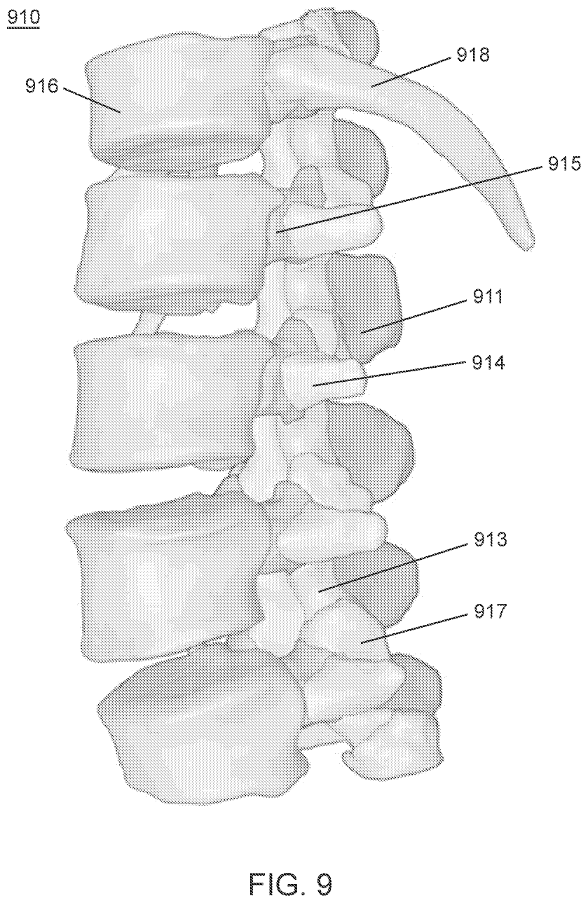

FIG. 6A is a flow chart of a vertebrae-based level identification process 520, according to embodiments. At 522, the image data (e.g., raw, processed, and/or distorted images) may be input into a segmentation model. In instances where the segmentation model is implemented as a CNN, the input images can be passed through the layers of the CNN. The segmentation model can return outputs on the image data, including segmentation outputs that identify different anatomical components in the image data. For example, depicted in FIG. 8C, the output 820 of the segmentation model can include the different anatomical parts or components denoted using different visual characteristics (e.g., different colors, patterns, or other characteristics). The different anatomical parts of a vertebra can include, for example, a spinous process 811, lamina 812, articular process 813, transverse process 814, pedicles 815, and/or vertebral body 816. As another example, FIG. 9 is a perspective view of a segmented 3D spine model where each vertebra corresponds to a level of the spine, and includes a vertebral body 916, pedicles 915, transverse processes 914, a lamina 913, articular processes 917, and a spinous process 911. In some embodiments, the segmentation output can also identify anatomical structures that neighbor the spine, such as, for example, a portion of a rib 918 extending from the spine, as depicted in FIG. 9. Suitable examples of segmentation are described in U.S. Patent Application Publication No. 2019/0105009, U.S. Patent Application Publication No. 2020/0151507, U.S. Patent Application Publication No. 2020/0410687, and U.S. Provisional Patent Application No. 63/187,777, incorporated above by reference.

Optionally, at 523, the output of the segmentation model may be postprocessed, e.g., using linear filtering (e.g., Gaussian filtering), non-linear filtering, median filtering, and/or morphological opening or closing. In some embodiments, postprocessing may include removing false positives after segmentation, determining whether some anatomical components are connected together (e.g., vertebral bodies or other portions of individual vertebrae) and, upon detection, disconnecting them, and/or smoothing the surface of anatomical components (including, for example, simplification of the geometry and/or filling holes in the geometry). For example, in case of a poor quality of an image scan or patients with spinal defects, vertebral bodies or other anatomical components of different levels may appear close to one another and/or overlapping (e.g., in contact with) each other. As such, the segmentation output obtained at 522 may show these components as being connected to each other. For instance, where a patient's L5 and S1 vertebral bodies are overlapping one another in an image, the segmentation output may show those vertebral bodies as being connected and therefore one anatomical component. In such instances, one or more postprocessing techniques, including, for example, a combination of watershed and distance transform algorithms, can be used to calculate separation lines between the two vertebral bodies.

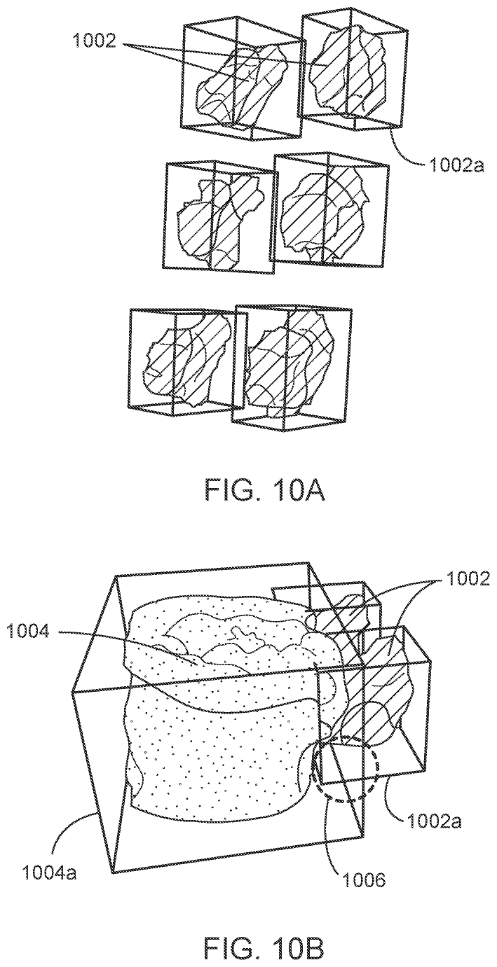

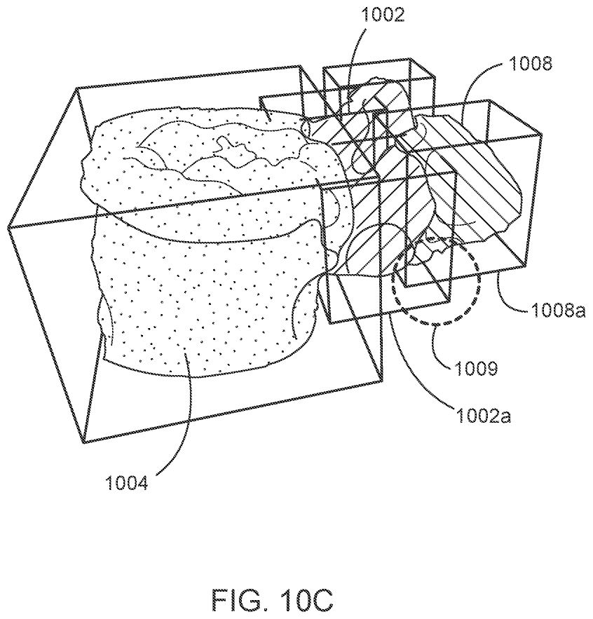

At 524, physical and geometrical parameters of the anatomical components of the spine (e.g., anatomical components of a set of vertebrae) can be determined. For example, geometrical parameters such as relative positions, morphological and spatial relationships, size, bounding volumes or information associated therewith (e.g., bounding box edges, bounding circle dimensions, etc.), and/or orientation based on the segmentation output(s) and/or the moment of inertia may be determined. For determining the levels of the vertebrae, the process 520 can analyze the anatomical components in each level in sequence. A first or starting anatomical component (or set of anatomical components) can be determined. In some embodiments, the starting anatomical component can be the pairs of pedicles of each vertebrae. For example, FIG. 10A depicts multiple sets of pedicles, with two pedicles belonging to each vertebral level. The pedicles of each level are generally distinct or separate from those of other levels, as shown in FIG. 10A. As such, the pedicles can be an advantageous anatomical component to start with, as it is unlikely that a pedicle pair would be incorrectly assigned to a vertebral level. Alternatively, the starting anatomical component can be a different part of a vertebra, such as, for example, the vertebral body (or a part of the vertebral body). In some cases, however, the vertebral bodies of patients may become joined, e.g., due to spinal defects. As such, it may be more desirable to start with a portion of the vertebrae that is more distinct and unlikely to overlap or be joined, such as, for example, the pedicles.

At 525, an initial level of the patient anatomy may be selected for processing. At 526, a first anatomical component (e.g., pedicles or vertebral body part) in the selected level may be determined. For example, as shown in FIG. 10A, a pair of pedicles 1002 for the selected vertebral level may be determined. A set of bounding boxes 1002a (or other bounding volumes) may be associated with the pair of pedicles 1002. In some embodiments, a determination of the pedicles that are associated with each vertebral level may be based on their location and relationship to other pedicles and/or anatomical components.

Then, for the selected spinal level, the anatomical components that are closest and/or intersecting with the first anatomical component can be assigned to that level. In particular, at 527, a second anatomical component (e.g., vertebral body part, pedicles) closest to and/or intersecting with the first anatomical component in the selected level may be identified or determined. For example, as shown in FIG. 10B, the bounding boxes 1002a of the pair of pedicles 1002 intersect with a bounding box 1004a of a vertebral body 1004, in one or more regions 1006. Given the intersection, the vertebral body 1004 can be assigned to the same level as the pedicles 1002.

At 528, additional anatomical component(s) (e.g., transverse processes, articular processes, spinous process, lamina) closest to and/or intersecting with the first or second anatomical components in the selected level may be identified or determined. For example, as shown in FIG. 10C, the bounding boxes 1002a of the pair of pedicles 1002 intersect with bounding boxes 1008a of articular processes 1008, in one or more regions 1009. Given the intersection, the articular processes 1008 can be assigned to the same level as the pedicles 1002 and the vertebral body 1004. Once the selected and intersecting and/or nearest anatomical components have been identified or determined, those components can be grouped together, e.g., as a subset of anatomical components. Once the anatomical components for the selected level have been assigned to that level, then the process 520 continues to 529, and if additional levels need to be processed (529: NO), then the process 520 returns to 525 to select the next level and repeats 526-528 for that next level. In some embodiments, when repeating 526-528, anatomical components that have already been assigned to a level can be excluded from assignment to later levels. Alternatively, when repeating 526-528, if an anatomical component that has already been assigned to another level is found to intersect with components of a different level, the process can discard that anatomical component or those levels from the level identification output. For example, in instances where significant overlap has occurred (e.g., during to poor image quality and/or spinal defects) and a particular anatomical component overlaps with and has been combined with another, such components and their associated levels can be discarded from the level identification output. Stated differently, the level identification output may not provide a level prediction for those anatomical components and/or levels.

The vertebrae-based level identification 520 ends when all desired levels of the vertebrae are processed. While 525-528 are described as a sequential process, it can be appreciated that the processes and steps described herein can be performed concurrently and/or as a combination of sequential and concurrent steps. The output of the vertebrae-based level identification 520 can include groupings of anatomical components to various levels and the morphological and spatial relationships between those anatomical components.

### ii. Disc-Based Level Identification

FIG. 6B is a flow chart of a disc-based level identification process 530, according to embodiments. The disc-based level identification process 530 can be similar to the vertebrae-based level identification process 520 described with reference to FIG. 6A, but the identification of various components in each level can be start from identifying the intervertebral discs between the vertebral bodies.

At 531, the image data (e.g., raw, processed, and/or distorted images) may be input into a segmentation model, similar to, for example, 522 in FIG. 6A. The segmentation model can return outputs on the image data, including segmentation outputs that identify different anatomical components in the image data. In some cases, a first segmentation model can be used to process the image data to identify anatomical components of a first type, at 531, and a second segmentation model can be used to process the image data to identify anatomical components of a second type, at 532. In such cases, the first segmentation model can be used to identify a first neighboring structure (and components thereof), and the second segmentation model can be used to identify an anatomical structure of interest (and components thereof). For example, a first segmentation model can be used to identify portions of the spine or the vertebrae (e.g., bone or not bone), and a second segmentation model can be used to identify the intervertebral discs and/or other soft tissue structures (e.g., nerves, muscle, etc.).

Optionally, at 533, the output of the segmentation model may be postprocessed, similar to, for example, 523 in FIG. 6A. At 534, physical and geometrical parameters of anatomical components of the intervertebral discs and vertebrae (and/or components thereof) can be determined. For example, geometrical parameters such as relative positions, size, bounding box edges, and orientation based on the segmentation output(s) and/or the moment of inertia may be determined.

After determining the physical and geometrical parameters of the intervertebral discs and vertebrae, the process 530 can analyze the anatomical components in each level in sequence. For example, at 535, an initial (or next) level (e.g., spine level) of the patient anatomy may be selected for processing. At 536, the intervertebral discs closest to the selected level (i.e., above (superior) and below (inferior) to a vertebrae level) may be identified or determined. At 537, the vertebral bodies between the intervertebral discs may be identified or determined. At 538, other anatomical components (e.g., pedicles, transverse processes, articular processes, spinous process, lamina) closest to (e.g., intersecting with) the vertebral body in the selected level may be identified or determined, e.g., in a process similar to 527-528 of FIG. 6A. Once the vertebral body and intersecting and/or nearest anatomical components have been identified or determined, those components can be grouped together, e.g., as a subset of anatomical components.

Once the anatomical components for the selected level have been assigned to that level, then the process 530 continues to 539, and if additional levels need to be processed (539: NO), then the process 530 returns to 535 to select the next level and repeats 536-538 for that next level. In some embodiments, when repeating 536-538, anatomical components that have already been assigned to a level can be excluded from assignment to later levels. Alternatively, when repeating 536-538, if an anatomical component that has already been assigned to another level is found to intersect with components of a different level, the process can discard that anatomical component or those levels from the level identification output.

The disc-based level identification 530 ends when all desired levels of the vertebrae are processed. While 535-538 are described as a sequential process, it can be appreciated that the processes and steps described herein can be performed concurrently and/or as a combination of sequential and concurrent steps. The output of the disc-based level identification 530 can include groupings of anatomical components (e.g., vertebrae and/or intervertebral discs) to various levels and the morphological and spatial relationships between those anatomical components.

### iii. Axial Image-Based Level Identification

FIG. 6C is a flow chart of an axial image-based level identification process 540, according to embodiments. The axial image-based level identification process 540 can utilize a level identification model, such as, for example, a CNN (e.g., CNN model 300) trained to predict vertebral level type and/or ordinal identifier based on axial image scans (e.g., axial CT scans) of spinal anatomy. The process 540 can be performed for each vertebra of the spine individually, with multiple scans of each vertebra of the spine being processed separately to provide level identification predictions. Alternatively, the process can be performed by processing multiple axial scans of the vertebrae together, e.g., as a sub-volume of a 3D volumetric image set.

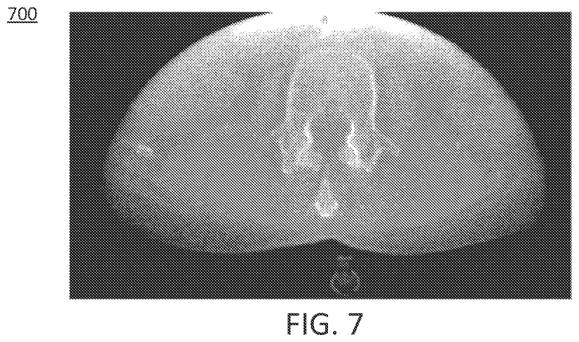

At 541, an initial vertebra of the patient anatomy may be selected, e.g., for predicting a level type or ordinal identifier. In some embodiments, the image data can include 3D volumetric image data that has multiple axial scans of each vertebra. At 542, an axial image associated with the selected vertebra may be selected. FIG. 7 provides an example of a 2D axial image 700 of a spine of a patient. The axial image 700 can be a CT scan of the spine. At 543, the selected axial image can be processed with a level identification model trained to identify level type (e.g., axial level identification model 236, CNN model 300). The level identification model can provide an output that can be used to assign a vertebral level type or vertebral level (ordinal identifier) to the spinal anatomy (or portions thereof) shown in the selected image. In some embodiments, the level identification model can output one or more probability maps that identifies the probability or likelihood that the image (or portions thereof) belongs to each level type class (e.g., sacrum(S), and/or sections of the spine including thoracic (T), lumbar (L), and/or cervical (C)). In some embodiments, the level identification model can output one or more probability maps that identifies the probability or likelihood that the image (or portions therefor) belongs to each level (ordinal identifier) class (e.g., T1-T12, L1-L5 (L6), S1-S5, and/or C1-C7). Stated differently, the output of the level identification model can include the per-class probabilities for each image (or portions thereof, e.g., pixel or groups of pixels). In particular, the level identification model can be configured to classify the image data into one of a plurality of classes (e.g., level types, ordinal identifiers). Accordingly, the level identification model can be configured to generate, for each image or portion thereof, the probability that that image or portion thereof belongs to any one of the classes from the plurality of classes. The plurality of classes can correspond to a plurality of level types (e.g., C, T, L, and/or S) or a plurality of vertebral levels or ordinal identifiers (e.g., T1-T12, L1-L5, S1-S5, and/or C1-C7).

At 544, a class (e.g., vertebral level type, vertebral level (ordinal identifier)) may be assigned to the axial image based on the output of the level identification model. For example, if any single class has a probability associated with it that is greater than a predefined threshold (e.g., greater than about 50 percent, about 60 percent, about 70 percent, about 80 percent, about 90percent, or about 99 percent, including all values and ranges therebetween) or has a probability associated with it that is significantly higher than the other classes (e.g., is at least a predefined amount or percentage greater than the other classes), then that class can be assigned to the selected axial image. Alternatively, where no single class has a probability associated with it that is greater than a predefined threshold or has a probability associated with it that is significantly higher than the other classes, then no class may be assigned to the axial image and that image can be discarded from the model prediction. FIGS. 8A and 8B are examples of 2D axial images 810, 830 of a spine of a patient, where a predicted level type has been assigned to each image 810, 830. For example, image 810 has been assigned a level type “Thoracic,” and image 830 has been assigned a level type “Sacrum.”

If more axial images are associated with the selected vertebra (545: NO), then the process 540 can return to 542 an iterative through the process with the next axial image associated with the selected vertebra. 542-544 can be repeated for each axial image until all of the axial images for the selected vertebra are processed (545: YES).

At 546, a class (e.g., level type, ordinal identifier) may be assigned to the selected vertebra based on the classes assigned to the axial images associated with the vertebra. For example, each vertebra may have a plurality of axial images associated with it. When processing each of the axial images (or groups of the axial images) with the level identification model, the model can return an output that can assign a particular class to that axial image or predict a particular class for that axial image. After all of the axial images have been processed, different axial images may have been assigned to different classes. For example, a set of axial images associated with a selected vertebra may include 80% that are assigned a first class (e.g., lumbar) and 20% that are assigned a second class (e.g., sacrum). In some embodiments, the class for the selected vertebra can be selected to be the class that has the greatest number of axial images assigned to it. As such, for a set of axial images of a vertebra where 80% are labeled “Lumbar” and 20% are labeled “Sacrum,” the vertebra can be assigned the level type “Lumbar.” Alternatively, other criteria (e.g., predefined number or percentage of axial images being associated with the class) can be used to determine the class that is assigned to the vertebra. The axial image-based level identification 540 ends when all of the desired vertebrae are processed (547: YES). Otherwise, the process returns to 541 and the next vertebra (and its associated axial images) is processed in 541-546.

While 541-547 are described as a sequential process, it can be appreciated that the processes and steps described herein can be performed concurrently and/or as a combination of sequential and concurrent steps. The output of the axial image-based level identification 540 can include vertebral level type assignments and/or vertebral level (ordinal identifier) assignments.

### iv. Sagittal or Coronal Image-Based Level Identification

FIG. 6D is a flow chart of a sagittal or coronal image-based level identification process 550, according to embodiments. The sagittal or coronal image-based level identification process 550 can be similar to the axial image-based identification process 540 described with reference to FIG. 6C, but the identification of level types or levels is based on processing sagittal or coronal images and using models (e.g., sagittal level identification model 238, coronal level identification model 239) trained to process these images. The output of the level identification can also be different and include, for example, a range of vertebral levels (ordinal identifiers) instead of a single vertebral level type or vertebral level (ordinal identifier).

At 551, a sagittal or coronal image depicting a set of vertebrae may be selected. Examples of sagittal views of the spinal anatomy are provided in FIGS. 12A and 13A. FIG. 12A is a 2D radiograph 1200 of a spine of a patient, taken from a sagittal view. FIG. 13A is a MRI image 1310 of a spine of a patient, taken as a sagittal section. Optionally, at 552, the selected image data (e.g., raw, processed, and/or distorted images) may be processed using one or more segmentation models. The segmentation model(s) can identify boundaries between neighboring anatomical components and/or distinguish between different types of anatomical components (e.g., vertebrae, intervertebral discs, nerves, muscles, etc.). Suitable examples of segmentation are described in U.S. Patent Application Publication No. 2019/0105009, U.S. Patent Application Publication No. 2020/0151507, U.S. Patent Application Publication No. 2020/0410687, and U.S. Provisional Patent Application No. 63/187,777, incorporated above by reference. FIG. 12B provides an example of a segmentation output 1210 associated with the X-ray image 1200 depicted in FIG. 12A. The output 1210 can be produced by processing the X-ray image 1200 with a segmentation model trained to segment X-ray images.

Optionally, at 553, the output(s) of the segmentation model(s) may be postprocessed, similar to, for example, 523 in FIG. 6A. And optionally, at 554, the output(s) of the segmentation model(s) may be merged with the sagittal or coronal image. FIG. 13B depicts an example of a merged image 1312 generated using the image 1310 of FIG. 13A and a segmentation output associated with the image. The segmentation output can be produced by processing the MRI image 1300 with a segmentation model trained to segment MRI images. A label map 1302 may be associated with the merged image 1312 and can include a plurality of labels each associated with a different anatomical component within the patient anatomy.

At 555, the selected sagittal or coronal image, optionally with the segmentation output (or a merged image of the sagittal or coronal image with the segmentation output), may be processed with a level identification model to generate an output. In some embodiments, the output of the level identification model can include one or more probability maps for each class (e.g., vertebral level or ordinal identifier) for each portion of the image. For example, the output of the level identification model can include the per-class probabilities for each pixel (or group of pixels) of each image of the image data. More specifically, the level identification model can be configured to classify the image data into one of a plurality of classes (e.g., vertebral levels or ordinal identifiers). Accordingly, the level identification model can be configured to generate, for each pixel or group of pixels in the images, the probability that that pixel or group of pixels belongs to any one of the classes from the plurality of classes. The plurality of classes can correspond to a plurality of vertebral levels or ordinal identifiers (e.g., T1-T12, L1-L5, S1-S5, and/or C1-C7).

At 556, a range of levels (e.g., vertebral levels or ordinal identifiers) may be assigned to the sagittal or coronal image based on the level identification model output. FIGS. 12C and 13C provide examples of levels that can be assigned to the set of anatomical components depicted in the X-ray image 1200 shown in FIG. 12A and the MRI image 1310 shown in FIG. 13A, respectively. FIG. 12C depicts a set of vertebral levels (ordinal identifiers) assigned to the different levels of the spine (e.g., T12, L1, L2, L3, L4, L5). FIG. 13C depicts ordinal identifiers assigned to vertebrae of the spine (e.g., T11, T12, L1, L2, L3, L4, L5) and the intervertebral discs between the vertebrae (e.g., T11/T12 Disc, T12/L1 Disc, L1/L2 Disc, L2/L3 Disc, L3/L4 Disc, L4/L5 Disc). While not depicted in FIGS. 12C or 13C, it can be appreciated that other types of anatomical structures (e.g., nerves, muscles, etc.) can also be assigned to one or more of the levels.

Steps 551-556 are repeated for each sagittal or coronal image until all of the sagittal or coronal images are processed (557: YES).

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto: inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which examples have been provided. The acts performed as part of the methods may be ordered in any suitable way. Accordingly, embodiments may be constructed in which acts are performed in an order different than illustrated, which may include performing some acts simultaneously, even though shown as sequential acts in illustrative embodiments.

As used herein, the terms “about” and/or “approximately” when used in conjunction with numerical values and/or ranges generally refer to those numerical values and/or ranges near to a recited numerical value and/or range. In some instances, the terms “about” and “approximately” may mean within +10% of the recited value. For example, in some instances, “about 100 [units]” may mean within #10% of 100 (e.g., from 90 to 110). The terms “about” and “approximately” may be used interchangeably.

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

Some embodiments and/or methods described herein can be performed by a different software (executed on hardware), hardware, or a combination thereof. Hardware modules may include, for example, a general-purpose processor, a field programmable gate array (FPGA), and/or an application specific integrated circuit (ASIC). Software modules (executed on hardware) can be expressed in a variety of software languages (e.g., computer code), including C, C++, Java™, Python, Ruby, Visual Basic™, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. For example, embodiments may be implemented using imperative programming languages (e.g., C, Fortran, etc.), functional programming languages (Haskell, Erlang, etc.), logical programming languages (e.g., Prolog), object-oriented programming languages (e.g., Java, C++, etc.), software libraries or toolkits (e.g., TensorFlow, PyTorch, Keras, etc.) or other suitable programming languages and/or development tools. Additional examples of computer code include, but are not limited to, control signals, encrypted code, and compressed code.

1. A method, comprising:
receiving image data of a set of anatomical components of an anatomical structure, the anatomical structure including a set of levels:
receiving segmentation data identifying the set of anatomical components in the image data:
implementing a first level identification process to generate a first set of level identification outputs, the first level identification process including determining geometrical parameters of the set of anatomical components based on the segmentation data and grouping the set of anatomical components into separate levels based on geometrical parameters of the set of anatomical components:
implementing a second level identification process to generate a second set of level identification outputs, the second level identification process including processing the image data of the set of anatomical components using a machine learning model to generate probability maps for each class of a plurality of classes associated with a set of level types or the set of levels:
assigning a level identifier of a level from the set of levels to each anatomical component from the set of anatomical components based on the first and second sets of level identification outputs; and
generating a visual representation of the anatomical structure including a visual depiction of the anatomical structure and visual elements indicative of the level identifiers assigned to the set of anatomical components.

2. The method of claim 1, wherein the anatomical structure is a spine, and the set of anatomical components includes a set of pedicle pairs and a set of vertebral bodies,
the implementing the first level identification process further including, for each pedicle pair from the set of pedicle pairs:
identifying, after determining the geometrical parameters of the set of anatomical components, a vertebral body from the set of vertebral bodies that intersects with or is nearest to the pedicle pair; and
subsequently identifying additional anatomical components from the remaining anatomical components from the set of anatomical components that intersect with or are nearest to that pedicle pair or the vertebral body,
the grouping the set of anatomical components into separate levels including grouping, for each pedicle pair from the set of pedicle pairs, the pedicle pair and the respective vertebral body and additional anatomical components into a level from the set of levels.

3. The method of claim 1, wherein the anatomical structure is a spine, and the set of anatomical components includes a set of intervertebral discs and a set of vertebral bodies,
the implementing the first level identification process further including, for each level from the set of levels:
identifying, after determining the geometrical parameters of the set of anatomical components, first and second intervertebral discs that are closest to the level:
identifying a vertebral body from the set of vertebral bodies that is disposed between the first and second intervertebral discs; and
subsequently identifying additional anatomical components from the remaining anatomical components from the set of anatomical components that intersect with or are nearest to the vertebral body:
the grouping the set of anatomical components into separate levels including grouping, for each level from the set of levels, the vertebral body and the respective additional anatomical components into the level.

4. The method of claim 1, wherein the implementing the first level identification process further includes:
iteratively performing until each anatomical component from the set of anatomical components has been grouped together with at least one other anatomical component from the set of anatomical components:
selecting, after determining the geometrical parameters of the set of anatomical components, an ungrouped anatomical component from the set of anatomical components; and
identifying additional ungrouped anatomical components from the set of anatomical components that intersect with or are nearest to the ungrouped anatomical component or subsequently identified ungrouped anatomical components,
the grouping the set of anatomical components into separate levels including grouping, after each iteration of selecting the ungrouped anatomical component and identifying the additional ungrouped anatomical components, the ungrouped anatomical component and the additional ungrouped anatomical components into a level from the set of levels.

5. The method of claim 4, wherein the determining the geometrical parameters of the set of anatomical components includes determining a bounding volume that contains each anatomical component,
the identifying the additional ungrouped anatomical components that intersects with or is nearest to the unassigned anatomical component or subsequently identified ungrouped anatomical components includes identifying one or more anatomical components that have a bounding volume that intersects with a bounding volume of the unassigned anatomical component or a subsequently identified ungrouped anatomical component.

6. The method of claim 1, wherein the anatomical structure is a spine, and the image data includes two-dimensional (2D) images of a three-dimensional volume containing a set of vertebrae of the spine,
the processing the image data using the machine learning model including processing, for each vertebra from the set of vertebrae, 2D images from the set of 2D images that contain at least a portion of the vertebra using the machine learning model to generate the probability maps for each of the 2D images that contain at least a portion of the vertebra:
the implementing the second level identification process further including, for each vertebra from the set of vertebrae:
assigning, for each of the 2D images containing at least a portion of the vertebra, at least one of a level type from the set of level types or a level identifier of a level from the set of levels based on the probability maps for the 2D image: and
determining, based on the level type or the level identifier assigned, a level type from the set of level types or a level identifier of a level from the set of levels for the vertebra.

7. The method of claim 6, wherein the 2D images include 2D axial scans of the set of vertebrae, and
the determining the level type or the level identifier for each vertebra from the set of vertebrae includes determining which level type or level identifier has been assigned to a greatest number of the 2D images that contain at least a portion of the vertebra.

8. The method of claim 1, wherein the anatomical structure is a spine, and the image data includes at least one two-dimensional (2D) image of a three-dimensional volume containing a set of vertebrae of the spine,
the processing the image data using the machine learning model including processing the at least one 2D image using the machine learning model to generate the probability maps for each of the at least one 2D image: and
the implementing the second level identification process further including determining, for each vertebra from the set of vertebrae, a level identifier of a level from the set of levels based on the probability maps for the at least one 2D image.

9. The method of claim 8, wherein the at least one 2D image includes a 2D sagittal scan or a 2D coronal scan of the set of vertebrae.

10. The method of any one of claims 6-9, wherein the machine learning model is a convolutional neural network trained to process 2D images of the set of vertebrae.

11. The method of any one of claims 6-10, wherein the set of 2D images includes at least one of: computed tomography (CT) images or magnetic resonance imaging (MRI) images.

12. The method of any one of claims 1-11, wherein the set of level types includes two or more of: sacrum, thoracic, lumbar, or cervical.

13. An apparatus, comprising:
a memory; and
a processor operatively coupled to the memory, the processor configured to:
receive image data of a set of anatomical components of an anatomical structure, the anatomical structure including a set of levels:
receive segmentation data identifying the set of anatomical components in the image data:
implement a first level identification process to generate a first set of level identification outputs, the first level identification process including determining geometrical parameters of the set of anatomical components and grouping the set of anatomical components into separate levels based on geometrical parameters of the set of anatomical components:
implement a second level identification process to generate a second set of level identification outputs, the second level identification process including processing the image data of the set of anatomical components using a machine learning model to generate probability maps for each class of a plurality of classes associated with a set of level types or the set of levels:
assign a level identifier of a level from the set of levels to each anatomical component from the set of anatomical components based on the first and second sets of level identification outputs; and
generate a visual representation of the anatomical structure including a visual depiction of the anatomical structure and visual elements indicative of the level identifiers assigned to the set of anatomical components.

14. The apparatus of claim 13, wherein the anatomical structure is a spine, and the set of anatomical components includes a set of pedicle pairs and a set of vertebral bodies,
the processor being configured to implement the first level identification process by further, for each pedicle pair from the set of pedicle pairs:
identifying, after determining the geometrical parameters of the set of anatomical components, a vertebral body from the set of vertebral bodies that intersects with or is nearest to the pedicle pair; and
subsequently identifying additional anatomical components from the remaining anatomical components from the set of anatomical components that intersect with or are nearest to that pedicle pair or the vertebral body,
the processor configured to group the set of anatomical components into separate levels by grouping, for each pedicle pair from the set of pedicle pairs, the pedicle pair and the respective vertebral body and additional anatomical components into a level from the set of levels.

15. The apparatus of claim 13, wherein the anatomical structure is a spine, and the set of anatomical components includes a set of intervertebral discs and a set of vertebral bodies,
the processor configured to implement the first level identification process by further, for each level from the set of levels:
identifying, after determining the geometrical parameters of the set of anatomical components, first and second intervertebral discs that are closest to the level:
identifying a vertebral body from the set of vertebral bodies that is disposed between the first and second intervertebral discs; and
subsequently identifying additional anatomical components from the remaining anatomical components from the set of anatomical components that intersect with or are nearest to the vertebral body:
the processor configured to group the set of anatomical components into separate levels by grouping, for each level from the set of levels, the vertebral body and the respective additional anatomical components into the level.

16. The apparatus of claim 13, wherein the processor is configured to implement the first level identification process by further:
iteratively performing until each anatomical component from the set of anatomical components has been grouped together with at least one other anatomical component from the set of anatomical components:
selecting, after determining the geometrical parameters of the set of anatomical components, an ungrouped anatomical component from the set of anatomical components: and
identifying additional ungrouped anatomical components from the set of anatomical components that intersect with or are nearest to the ungrouped anatomical component or subsequently identified ungrouped anatomical components,
the processor configured to group the set of anatomical components into separate levels by grouping, after each iteration of selecting the ungrouped anatomical component and identifying the additional ungrouped anatomical components, the ungrouped anatomical component and the additional ungrouped anatomical components into a level from the set of levels.

17. The apparatus of claim 16, wherein the processor is configured to determine the geometrical parameters of the set of anatomical components by determining a bounding volume that contains each anatomical component,
the processor configured to identify the additional ungrouped anatomical components that intersects with or is nearest to the unassigned anatomical component or subsequently identified ungrouped anatomical components by identifying one or more anatomical components that have a bounding volume that intersects with a bounding volume of the unassigned anatomical component or a subsequently identified ungrouped anatomical component.

18. The apparatus of claim 13, wherein the anatomical structure is a spine, and the image data includes two-dimensional (2D) images of a three-dimensional volume containing a set of vertebrae of the spine,
the processor configured to process the image data using the machine learning model by processing, for each vertebra from the set of vertebrae, 2D images from the set of 2D images that contain at least a portion of the vertebra using the machine learning model to generate the probability maps for each of the 2D images that contain at least a portion of the vertebra:
the processor configured to implement the second level identification process by further, for each vertebra from the set of vertebrae:
assigning, for each of the 2D images containing at least a portion of the vertebra, at least one of a level type from the set of level types or a level identifier of a level from the set of levels based on the probability maps for the 2D image; and
determining, based on the level type or the level identifier assigned, a level type from the set of level types or a level identifier of a level from the set of levels for the vertebra.

19. The apparatus of claim 13, wherein the anatomical structure is a spine, and the image data includes at least one two-dimensional (2D) image of a three-dimensional volume containing a set of vertebrae of the spine,
the processor configured to process the image data using the machine learning model by processing the at least one 2D image using the machine learning model to generate the probability maps for each of the at least one 2D image; and
the processor configured to implement the second level identification process by further determining, for each vertebra from the set of vertebrae, a level identifier of a level from the set of levels based on the probability maps for the at least one 2D image.

20. A method, comprising:
receiving a set of two-dimensional (2D) images of a three-dimensional volume containing a set of anatomical components of an anatomical structure, the anatomical structure including a set of levels, the set of 2D images including subsets of 2D images each associated with a different anatomical component from the set of anatomical components:
for each anatomical component from the set of anatomical components:
processing, using a convolutional neural network (CNN) trained to identify the set of levels or level types of the set of levels, each 2D image from the subset of 2D images associated with the anatomical component to output a predicted level or level type for the anatomical component based on the 2D image; and
assigning a level or a level type to the anatomical component based on the predicted levels or level types for the anatomical component output by processing the subset of 2D images associated with the anatomical component; and
generating a visual representation of the anatomical structure including a visual depiction of the anatomical structure and visual elements indicative of the level or level type assigned to the set of anatomical components.

21. The method of claim 20, wherein the anatomical structure is a spine, and the set of anatomical components includes a set of vertebrae of the spine.

22. The method of any one of claims 20-21, wherein each vertebra from the set of vertebrae of the spine is associated with one of a set of level types including two or more of: sacrum, thoracic, lumbar, or cervical.

23. The method of any one of claims 20-22, wherein the predicted levels or level types for at least one anatomical component includes a set of predicted levels or level types, and the assigning the level or the level type to each anatomical component from the set of anatomical components includes, for each of the at least one anatomical component:
determining a percentage or number of the subset of 2D images associated with the anatomical component that has each of the set of predicted levels or level types: and
in response to a predicted level or level type of the set of predicted levels or level types having a greater percentage or number than the remaining of the set of predicted levels or level types, assigning the predicted level or level type having the greater percentage or number as the level or level type of the anatomical component.

24. The method of any one of claims 20-22, wherein the predicted levels or level types for at least one anatomical component includes a set of predicted levels or level types, and the assigning the level or the level type to each anatomical component from the set of anatomical components includes, for each of the at least one anatomical component:
determining a percentage or number of the subset of 2D images associated with the anatomical component that has each of the set of predicted levels or level types; and
in response to a single predicted level or level type of the set of predicted levels or level types having a percentage or number greater than a predetermined threshold, assigning the single predicted level or level type as the level or level type of the anatomical component.

25. The method of any one of claims 20-23, wherein the predicted levels or level types for at least one anatomical component includes a single predicted level or level type, and the assigning the level or the level type to each anatomical component from the set of anatomical components includes assigning the single predicted level or level type as the level or level type of the at least one anatomical component.

26. The method of any one of claims 20-25, wherein the set of 2D images includes at least one of: computed tomography (CT) images or magnetic resonance imaging (MRI) images.

27. An apparatus, comprising:
a memory: and
a processor operatively coupled to the memory, the processor configured to:
receive a set of two-dimensional (2D) images of a three-dimensional volume containing a set of anatomical components of an anatomical structure, the anatomical structure including a set of levels, the set of 2D images including subsets of 2D images each associated with a different anatomical component from the set of anatomical components:
for each anatomical component from the set of anatomical components:
process, using a convolutional neural network (CNN) trained to identify the set of levels or level types of the set of levels, each 2D image from the subset of 2D images associated with the anatomical component to output a predicted level or level type for the anatomical component based on the 2D image; and
assign a level or a level type to the anatomical component based on the predicted levels or level types for the anatomical component output by processing the subset of 2D images associated with the anatomical component: and
generate a visual representation of the anatomical structure including a visual depiction of the anatomical structure and visual elements indicative of the level or level type assigned to the set of anatomical components.

28. The apparatus of claim 27, wherein the anatomical structure is a spine, and the set of anatomical components includes a set of vertebrae of the spine.

29. The apparatus of any one of claims 27-28, wherein each vertebra from the set of vertebrae of the spine is associated with one of a set of level types including two or more of:
sacrum, thoracic, lumbar, or cervical.

30. The apparatus of any one of claims 27-29, wherein the predicted levels or level types for at least one anatomical component includes a set of predicted levels or level types, and the processor is configured to assign the level or the level type to each anatomical component from the set of anatomical components by, for each of the at least one anatomical component:
determining a percentage or number of the subset of 2D images associated with the anatomical component that has each of the set of predicted levels or level types: and
in response to a predicted level or level type of the set of predicted levels or level types having a greater percentage or number than the remaining of the set of predicted levels or level types, assigning the predicted level or level type having the greater percentage or number as the level or level type of the anatomical component.

31. The apparatus of any one of claims 27-29, wherein the predicted levels or level types for at least one anatomical component includes a set of predicted levels or level types, and the processor is configured to assign the level or the level type to each anatomical component from the set of anatomical components by, for each of the at least one anatomical component:
determining a percentage or number of the subset of 2D images associated with the anatomical component that has each of the set of predicted levels or level types; and
in response to a single predicted level or level type of the set of predicted levels or level types having a percentage or number greater than a predetermined threshold, assigning the single predicted level or level type as the level or level type of the anatomical component.

32. The apparatus of any one of claims 27-29, wherein the predicted levels or level types for at least one anatomical component includes a single predicted level or level type, and the processor is configured to assign the level or the level type to each anatomical component from the set of anatomical components by assigning the single predicted level or level type as the level or level type of the at least one anatomical component.

33. The apparatus of any one of claims 27-32, wherein the set of 2D images includes at least one of: computed tomography (CT) images or magnetic resonance imaging (MRI) images.
